(19) United States
(12) Patent Application Publication
(10) Pub. No.: US 2006/0122529 A1
(43) Pub. Date: Jun. 8, 2006

(54) Low noise amplifier for electro-physiological signal sensing

(76) Inventor: Yang Tsau, Bethesda, MD (US)

Correspondence Address: BERENATO, WHITE & STAVISH, LLC, 6550 ROCK SPRING DRIVE, SUITE 240, BETHESDA, MD, 20817, US

(21) Appl. No.: 11/004,641

(22) Filed: Dec. 6, 2004

(51) Int. Cl. A61B 5/04

(52) U.S. Cl. 600/544

(57) ABSTRACT

A reliable, safe, accurate, low noise, inexpensive, portable amplifier circuit is adapted to accurately amplify both AC and DC neural response signals. A patient or subject is electrically connected to a multi-channel system for electrically measuring the patient's AC and DC neural response signals at a plurality of locations using electrodes connected through a multi-electrode cable. The neural response signals are input to a digital DC amplifier to filter, amplify and digitize the neural response signals. Digitized neural response signals are converted to optical signals and transmitted via a fiber optic cable to an interface that is preferably connected to a patient stimulus generator (e.g., a Ganzfeld stimulator or pattern stimulator for multi-focal ERG). The system also includes a stand-alone computer such as an IBM® compatible Personal Computer (PC) for two-way communication with the interface via a standard data interface cable (e.g., a USB cable). In the preferred embodiment, a digital DC amplifier is worn by the patient and receives each neural response signal at a two-conductor balanced input; a surge suppression circuit limits excessive voltage transients at the input. The neural response signal is next input to a balanced buffer amplifier stage for impedance matching and the buffered neural response signal is then input to a balanced, adjustable pre-amplifier stage having an adjustable gain which can be varied (e.g., from ×1 to ×64). The buffered, amplified neural response signal is then digitized for storage in a memory and transmission to a fiber-optic digital transmission circuit. An adjustable impedance element generates a DC offset compensation signal used to control a D.C. offset compensation amplifier to generate an offset control signal for input to gain-adjustable pre-amplifier stage, to maximize sensitivity and usable dynamic range.

A computer program listing appendix is submitted herewith on compact disc recordable (CD-R) as Appendix A. Duplicate copies of Appendix A are provided as Copy 1 and Copy 2. The materials on the CD-R are identical to each other.

The files on the compact discs are incorporated herein by reference, and are listed below:

| File Name | Size in bytes | Date |
|---|---|---|
| C_code_DCamp.txt | 50.4 Kb | Oct. 29, 2003 |
| C_code_Interface.txt | 35.8 Kb | Oct. 29, 2003 |

### BACKGROUND OF THE INVENTION

1. Field of the Invention

This invention relates generally to instruments for use in diagnosis and assessment of medical disorders by objective means. More specifically, it relates to diagnostic testing and, more particularly, to diagnostic testing of the nervous system. In humans, such testing is often done in conjunction with diagnostic procedures such as electro-retinography (ERG) and multi-focal ERG.

2. Discussion of the Prior Art

ERG provides for the determination of extent and magnitude of local defects in the visual field of the eye by simultaneous stimulation of a large number of locations on the retina and analysis of the elicited electrical signals derived from the eye or the scalp (this procedure is also known as visual evoked cortical response or VECR). Electrically measured neural response signals derived from such procedures are very low in amplitude and are susceptible to corruption in noise.

Time is a factor in successful diagnostic testing, and 10 to 20 minutes may be all the time one may have for completing a testing regimen, since even for a given patient, responses may vary from day to day. Diagnostic testing instruments must also accommodate artifactual noise, such as produced by blinks, which affect a test subject's measured neural responses to a degree.

Diagnostic testing on human subjects must also be safe, and so many jurisdictions require that a human test subject be electrically isolated from any possible exposure to excessive electric voltages or currents. In prior art diagnostic instruments, this mandated the use of hospital grade isolation transformers, which are bulky, expensive and prone to introducing electrical noise artifacts in an electrically measured neural response signal.

In certain nervous system diagnostic test procedures, an electrically measured neural response signal may have a rapidly time-varying component with an amplitude of approximately one to ten microvolts (1-10 μV), and may have a slowly varying Direct Current (DC) offset of approximately ten millivolts (10 mV). Amplifiers adapted for use in diagnostic instruments have traditionally dealt with this relatively enormous DC offset by essentially discarding it, and have employed series capacitors selected to permit coupling of only the rapidly time-varying component of an electrically measured neural response signal. Otherwise, the amplifiers would be prone to saturation and non-linear response. The prior art practice of omitting the electrically measured neural response signal's slowly varying Direct Current (DC) offset component may be the primary cause for a lack of basic science tending to promote understanding of slowly varying neural responses.

As shown in FIGS. 1 and 2, a patient, 30 wears electrodes and is electrically connected through a heavy and expensive electrical isolation transformer 32 to a multistage filtering amplifier circuit 34 which generates an amplified neural response signal for input to an analog to digital converter 36. The digitized neural response signal is then provided to a computer or CPU 38 for data logging. CPU 38 also controls the diagnostic testing procedure and so provides one or more control signals to a stimulus 40 (e.g., a Ganzfeld stimulator or pattern stimulator for multi-focal ERG).

Amplifier circuits used in diagnostic instruments for sensing electrically measured neural response signals have traditionally included several active stages of buffering, amplification and filtering, with one or more stages being capacitively coupled to block the undesired DC components of the electrically measured neural response signal. One popular configuration (e.g., 34) includes a first buffer amplifier stage 42 for impedance matching which generates a buffered signal that is input to a preamplifier stage 44 having a gain of approximately one thousand (1000); the output of preamplifier stage 44 is capacitively coupled (via capacitor 46a) to a low cut variable frequency adjustable filter stage 48, with a low-cut frequency range of zero to one cycle per second or hertz (Hz), and the low cut filter stage's output is input to a notch filter stage 50, usually to remove the power supply related noise signal (e.g., at 60 Hz for the US). The notch filter stage's output is input to a high-cut adjustable frequency filter stage 52, having a high cut frequency set to approximately 100 Hz, and the high-cut stage output is capacitively coupled (via capacitor 46b) to an adjustable gain amplifier stage 54. The adjustable gain amplifier stage's output is then capacitively coupled (via capacitor 46c) to an Analog to Digital converter (ADC) 36 so that an amplified, digitized, neural response signal is available to computer or CPU 38 for further analysis (e.g., thresholding to detect a selected event) or for display.

Prior art amplifier circuits such as that shown in FIGS. 1 and 2 have not been entirely satisfactory for every neural testing application, however, because they are not sensitive enough to detect many weak electrical signals. Signal to Noise ratio (S/N) is a figure of merit for instrument amplifiers, where noise should be minimized. Each amplification stage and the electrical connections between the stages contribute to background noise tending to corrupt the electrically measured neural response signal. As noted above, the electrically measured neural response signal's rapidly time-varying component can have an amplitude of approximately one to ten microvolts (1-10 μV). The noise specifications for the prior art amplifiers adapted for use in diagnostic instruments have traditionally been less than three microvolts (3 μV), and so the smaller signals (e.g., 1-3 microvolts) are lost in the amplifier's noise and remain unavailable for analysis or display.

Turning now to a more general view, all physiological functions are accompanied by electrical activities that can be recorded and measured using an electronic device. Usually, the electrical activities accompanying physiological functions can be detected with amplifier circuits having millivolt (mV) to microvolt (uV) sensitivity, because the electrically measured neural response signals range at very low amplitude from the mV range to below one uV.

Large signal physiological phenomena, such as changes in Blood Pressure, muscle contractions or changes in intracellular and extracellular ion concentration can provide relatively large electrical signals when using specially adapted transducers, but the signals usually vary slowly (and so signal bandwidth varies from DC to less than 10 Hz). In addition, these large, slow signals tend to have a small fraction (0.001%-10%) of AC components as compared to their DC component.

For smaller electrically measured neural response signals, such as those sensed in spontaneous EEG, event evoked potential, and focal/multi-focal ERG signals, the amplitude is approximately 5 uV or less. Some signal components such as “a wave” and “b wave” in an ERG recording are relatively large, with values ranging from 20 uV to 200 uV, and the “c wave” is small (about 5 uV) and very slow (0.01 Hz and below).

Researchers have recently expressed an increased interest in very slowly changing or DC-ERG recordings. Some signals, such as membrane potentials, action potentials and single ion channel activities have signal sizes in the mV range and have a bandwidth of interest from DC to approximately 10 kHz. Therefore, in order to meet the requirements of amplification for all of these physiological signals, an ideal amplifier for electrically measured neural response signals would, ideally have an effective frequency range from DC to 10 kHz and would provide sensitivity to be able to amplify signals down to less than one microvolt. Accordingly, the noise level for an ideal amplifier would necessarily be lower than the electrically measured neural response signal's level and so would ideally be less than 1.0 uV.

This ideal amplifier is not found in the prior art. The currently available amplifiers often utilize an AC/DC separation circuit (e.g., a DC blocking series capacitor) to obtain (1) AC components for further amplification and (2) DC components for measurement The Grass™ brand amplifier model 12 and the LKC™ brand patient amplifier are exemplary of the prior art. The AC/DC separation frequency is usually in the range of 0.001-0.1 Hz. The lowest frequency or DC component is relatively easy to deal with. However, the AC components, especially the lower frequency AC components, are not always suitable. If the AC/DC separation frequency is selected to be too high (e.g., 0.1 Hz or higher), the slower components of the electrically measured neural response signal will be cut off. If the AC/DC separation frequency is set too low (e.g., close to 0.001 Hz or even lower), any large transient input fluctuation due to spurious artifacts (such as patient's head movement during ERG/EOG/VEP recording) will temporarily saturate and disable the amplifier and cause prolonged amplifier recovery time (from a few seconds to minutes). This is not practical for a DC amplifier. An ideal DC amplifier should have zero recovery time with high sensitivity to small signals.

Once the electrically measured neural response signals are amplified, they are fed to the recording devices, measurement devices, and/or analysis devices. It is common that the neural response signals are first digitized by analog to digital conversion (ADC) for recording, measurement, and analysis. ADC for DC and low frequency signals is tedious while that for high frequency signals has been a challenge. Fortunately, physiological signals can be digitized by the progressive approximation converters, which can easily reach the speed of 10 kHz. However, this type of converter usually has relatively low conversion (8-16 bits, typically 12 bits) resolution, which limits the dynamic range of the digitized data. For example, a 12 bit ADC (e.g., 36 as shown in FIG. 1) having input range of +/−one volt (1V) can detect signals so long as they are greater than or equal to 0.5 mV. For this detection level, in order to detect a signal having an amplitude of 0.1 uV, an amplifier with a voltage gain of at least five thousand (5000) would be required to provide an ADC input signal in the range of 0.5 mV. Furthermore, to avoid the “bit noise” generated by ADC 36, it is necessary to increase the amplification gain, and so the dynamic range of input is further reduced. In other words, the DC tolerance for the sensitivity of 0.1 uV is less than 0.2 mV. This is not acceptable for signals with DC drift more than 0.2 mV, such as those bioelectrical signals recorded using electrodes, where the DC drift can be up to 10 mV.

Another problem confronted by those attempting to measure electrically measured neural response signals is electrical noise, which may come from the amplifier circuit 34 or from external interference. Ultra-low noise instrument amplifiers have become available recently, but these ultra-low noise amplifier circuits haven't been optimized for physiological signal recording applications. Avoiding external interference ordinarily requires careful design and construction, but is important because noise can dramatically affect the accuracy of recordings for weak physiological signals (e.g., at a uV level). Introduction of external noise is almost inevitable because of the physical connection between the signal source (the patient) and the noise sources (e.g., the environment, the computer interface, the power supply, etc.). Although isolation amplifiers are required by law in physiological signal amplification, AC noise can still be coupled to the amplifier input and output, causing superposition of the noise on the desired neural response signal.

As a result of these challenges, prior art amplifiers have become increasingly bulky and expensive and require higher parts counts, high quality parts and extensive noise shielding. Bulky amplifiers take up excessive space in a medical facility and restrict the patient's or subject's movement.

There is a need, therefore, for an inexpensive amplifier circuit which overcomes these challenges and permits portable, reliable, safe, accurate, low noise amplification of electrically measured AC and DC neural response signals.

### OBJECTS AND SUMMARY OF THE INVENTION

Accordingly, it is a primary object of the present invention to overcome the above mentioned difficulties by providing a reliable, safe, accurate, low noise, inexpensive, portable amplifier circuit adapted to accurately amplify neural response signals.

Another object of the present invention is accurately sensing both AC and DC neural response signals.

Yet another object of the present invention is providing an amplifier circuit that is not bulky and is easily worn or carried by the patient.

Yet another object of the present invention is providing a safe amplifier circuit that clearly poses no risk of electrocution to the patient.

Another object of the present invention is providing an ultra-low noise, highly dynamic digital DC amplifier with a digital interface that is superior to prior art amplifiers.

Yet another object of the present invention is providing support for multiple channels with multi-channel fiber-optic (and therefore isolated) digital data links.

The aforesaid objects are achieved individually and in combination, and it is not intended that the present invention be construed as requiring two or more of the objects to be combined unless expressly required by the claims attached hereto.

As noted above, an ideal DC amplifier should have zero recovery time with high sensitivity to small neural response signals; this objective is met by a true DC amplifier with no AC signal separation. In the amplifier of present invention, minimal recovery time is achieved by unbiasing the DC offset. “DC” neural response signals, as used in the context of the present invention, refers to signals having very long time constants or varying periodically with very low frequencies (e.g., on the order of one tenth Hertz (0.1 Hz)), and “AC” signals are, in relative terms, signals having shorter time constants or varying periodically with higher frequencies.

In accordance with the present invention, a patient is electrically connected to a system for electrically measuring the patient's AC and DC neural response signals. The system preferably has a plurality of channels (e.g., four) for sensing the patient's neural responses at a plurality of locations using electrodes connected through a multi-electrode cable. The neural response signals are input to a digital DC amplifier which filters, amplifies and digitizes the neural response signals. Digitized neural response signals are converted to optical signals and transmitted via a fiber optic cable to an interface or controller which may be connected to a patient stimulus generator (e.g., a Ganzfeld stimulator or pattern stimulator for multi-focal ERG). The system also includes a stand-alone computer such as an IBM or Apple compatible Personal Computer (PC) for two-way communication with the interface via a standard data interface cable (e.g., a USB cable).

In the preferred embodiment, a digital DC amplifier worn by the patient receives the neural response signal at a two-conductor balanced input, and a surge suppression circuit limits excessive voltage transients at the input. The neural response signal is next input to a balanced buffer amplifier stage for impedance matching and the buffered neural response signal is then input to a balanced, adjustable pre-amplifier stage having an adjustable gain which can be varied (e.g., from ×1 to ×64). The buffered, amplified neural response signal is then input to an Analog to Digital Converter (ADC) which provides a digitized neural response signal to a microprocessor for storage in a memory and transmission to a fiber-optic digital transmission circuit. The microprocessor is also connected to and responsive to a fiber-optic digital receiving circuit. Part of the pre-programmed process executed by the microprocessor includes control of an adjustable impedance element also known as a digital potentiometer for generating a DC offset compensation signal used to control a D.C. offset compensation amplifier, preferably over a range of plus or minus one volt, to generate an offset control signal for input to gain-adjustable pre-amplifier stage. Another part of the pre-programmed process executed by the microprocessor includes control of an adjustable impedance testing circuit which provides an impedance test signal to the input of the Digital D.C. amplifier.

A problem confronted by those attempting to electrically measure neural response signals is electrical noise, which may come from the amplifier circuit 34 or from external interference. Ultra-low noise instrument amplifiers become available recently (e.g., the TH4130 amplifier by Texas Instruments™), and these new amplifiers have noise levels as low as 10 nV/Hz-2 but haven't been optimized for physiological signal recording applications which require patient isolation and some means to deal with external noise or other interference.

In the amplifier of the present invention, effects from external interference are minimized by careful design; it has been discovered that when amplifying neural response signals, the major external noise source is from Common Mode Noise (CMN), effects from which are minimized through use of differential inputs to the amplifier stages. Preferably, differential amplification is employed through all the stages including the analog filter circuits to the computer interface. CMN noise may be superimposed on the desired signal when using single ended amplifiers, as are often used in currently available biomedical instruments. Even if the noise signal amplitude is relatively small (e.g. a few millivolts (mV)), the noise signal may still tremendously affect the quality of recording for weak physiological signals (e.g., at the microvolt (uV)) level.

The above and still further objects, features and advantages of the present invention will become apparent upon consideration of the following detailed description of a specific embodiment thereof, particularly when taken in conjunction with the accompanying drawings, wherein like reference numerals in the various figures are utilized to designate like components.

### BRIEF DESCRIPTION OF THE DRAWINGS

FIG. 1 is a diagram illustrating a system for electrically measuring neural response signals, in accordance with the prior art.

FIG. 2 is a schematic diagram illustrating an amplifier circuit for use in the system of FIG. 1, in accordance with the prior art.

FIG. 3 is a diagram illustrating a system for electrically measuring both AC and DC neural response signals, in accordance with the present invention.

FIG. 4 is a block diagram illustrating an amplifier circuit for use in the system of FIG. 3, in accordance with the present invention.

FIGS. 5a, 5b and 5c are schematic diagrams of a first embodiment of the amplifier circuit of FIGS. 3 and 4, in accordance with the present invention.

FIGS. 6a and 6b are schematic diagrams of a second embodiment of the amplifier circuit of FIGS. 3 and 4, in accordance with the present invention.

FIGS. 7a and 7b are schematic diagrams of an interface control circuit for use in the system of FIG. 3, in accordance with the present invention.

FIG. 8 is a schematic diagram of a multi-channel embodiment of the amplifier circuit of FIGS. 3 and 4, in accordance with the present invention.

FIGS. 9a through 9f are a process flow diagram illustrating pre-programmed method steps executed using the microprocessor in the wearable DC amplifier unit of FIG. 4.

FIGS. 10a through 10g are a process flow diagram illustrating pre-programmed method steps executed by the interface unit used to communicate with and control the wearable DC amplifier unit.

FIG. 11 is process flow diagram illustrating pre-programmed method steps executed by the user's personal computer when used to communicate with and control the wearable DC amplifier unit through the interface unit.

FIG. 12 is a diagram illustrating the layout of fiber-optic communication elements within the wearable DC amplifier unit, in accordance with the present invention.

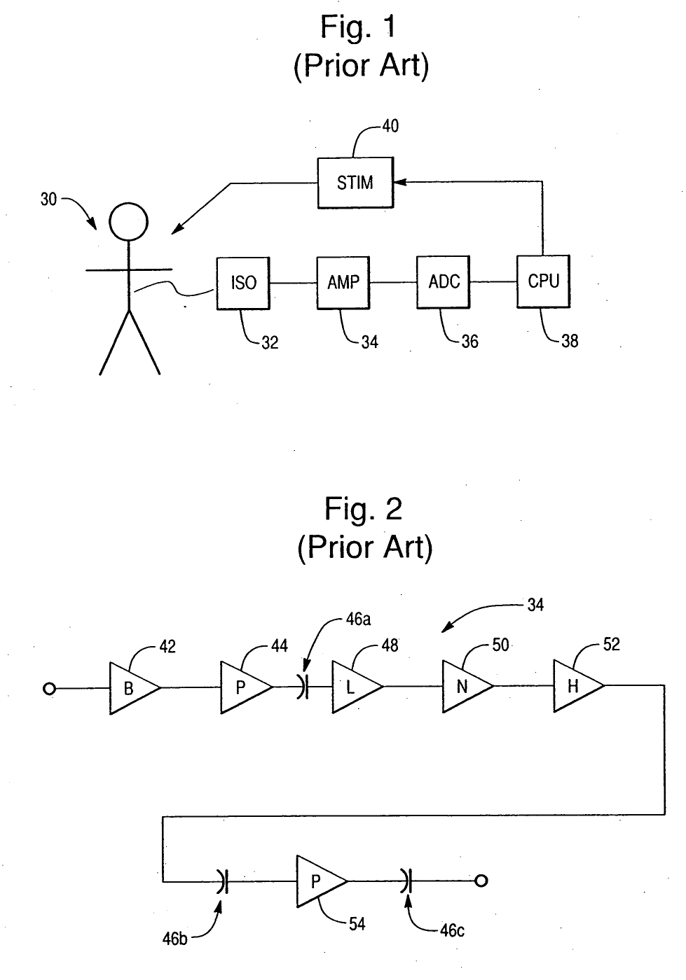

### DESCRIPTION OF THE PREFERRED EMBODIMENT

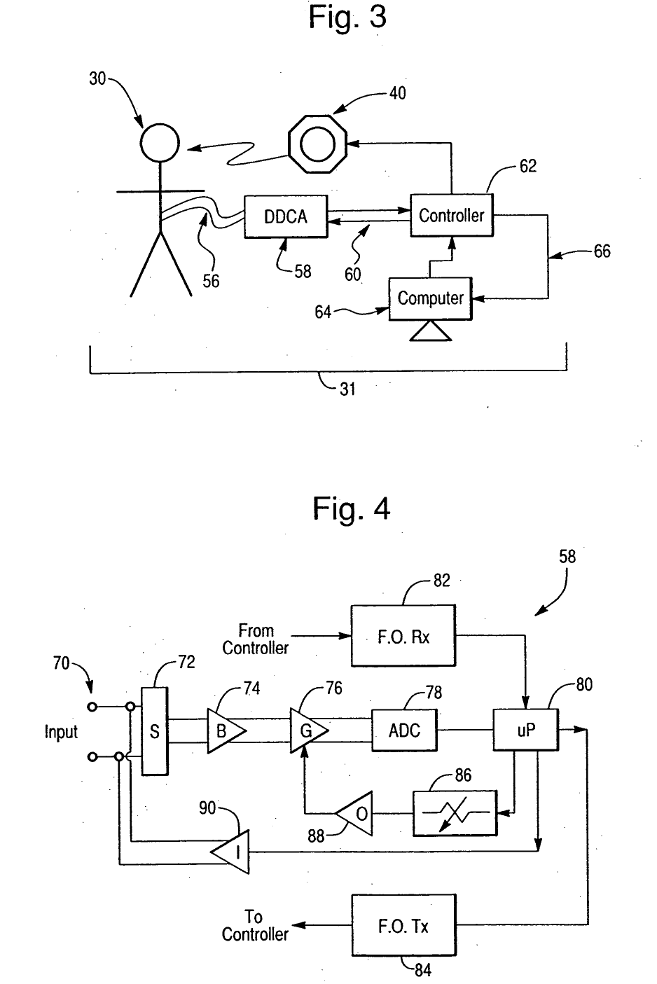

As best seen in FIGS. 3 and 4, a patient or subject 30 is electrically connected to a system 31 for electrically measuring a patient's AC and DC neural response signals. System 31 preferably has a plurality of channels (e.g., four) for sensing the patient's neural responses at a plurality of locations on the body using electrodes connected to a multi-electrode cable 56. The neural response signals are input to a wearable digital DC amplifier 58 which filters, amplifies and digitizes the neural response signals. Digitized neural response signals are converted to optical signals are transmitted via fiber optic cable 60 to a interface controller 62 which is also optionally connected to a patient stimulus generator 40 (e.g., a Ganzfeld stimulator or pattern stimulator for multi-focal ERG). System 31 also preferably includes a stand-alone computer such as an IBM™ brand compatible Personal Computer (PC) 64 for two-way communication with interface controller 62, preferably via a standard data interface cable (e.g., a USB cable).

Referring now to FIG. 4, digital DC amplifier 58 receives the neural response signal at a two-conductor balanced input 70, and a surge suppression circuit 72 limits excessive voltage transients at the input. The neural response signal is next input to a balanced buffer amplifier stage 74 for impedance matching and the buffered neural response signal is then input to a balanced gain-adjustable pre-amplifier stage 76 having an adjustable gain which can be varied (e.g., from ×1 to ×64). The buffered, amplified neural response signal is then input to an Analog to Digital Converter 78 which provides a digitized neural response signal to a microprocessor 80 for storage in memory and transmission to a fiber-optic digital transmission circuit 84.

Microprocessor 80 is connected to and responsive to a fiber-optic digital receiving circuit 82 and fiber-optic digital transmitting circuit 84, transmitting data to and receiving data from interface controller 62 via this bi-directional fiber optic link.

Part of the pre-programmed process executed by microprocessor 80 includes control of an adjustable impedance element also known as a digital potentiometer 86 for generating a DC offset compensation signal used to control a D.C. offset compensation amplifier 88, preferably over a range of plus or minus one volt, to generate an offset control signal for input to gain-adjustable pre-amplifier stage 76. Another part of the pre-programmed process executed by microprocessor 80 includes control of an adjustable impedance testing circuit 90 which provides an impedance test signal to the input 70 of Digital D.C. amplifier 58.

Wearable DC Amplifier System Considerations:

Turning now to a more system-wide perspective, in order to overcome the problems with ADCs of the prior art (e.g., the bit noise and limited dynamic range of progressive approximation converters), an alternative approach has been found. Delta-sigma ADC devices can be selected with higher resolution (20-26 bits, typically 24 bits). The dynamic range of input for 0.1 uV minimum signal detection for a 20 bit AD converter, for example, is +/−50 mV. Therefore, signals with different dynamic ranges can be recorded simultaneously without changing the amplification gain. An application example is to simultaneously record full field ERG (up to 1 mV) and focal ERG (around 1 uV) with the same gain setting. Furthermore, the DC drift from electrodes is relatively easily tolerated if it is within +/−50 mV. To further compensate for signals of widely varying level (e.g., with a DC component of +/−5V), the DC offset control circuit including digital potentiometer 86 and offset compensation amplifier 88 provides a wide range of automatic adjustment. While traditional Delta-Sigma ADC devices tend to allow only slow sampling rates, typically under 10 samples/sec, newer delta-sigma ADC devices have been demonstrated at sampling rates up to 40,000 samples/sec for 19 bit resolution.

As noted above, low noise level is critical for the success of the digital DC amplifier 58 of the present invention. Noise may come from the amplifier circuit or from external interference sources. Ultra-low noise instrument amplifiers such as TH4130 (from Texas Instruments), have a noise level as low as 10 nV/Hz-2. To date, such ultra-low noise amplifier circuits haven't been used in instruments for making physiological signal recordings.

One aspect of the present invention is minimizing external noise and interference signals through careful design. A major external noise source is so called Common Mode Noise (CMN), which is preferably minimized by using differential inputs to amplifier stages. This requires the differential amplification throughout all the stages (e.g., 74, 76) including analog filter circuits to the computer interface controller 62. Harmful noise may be superimposed on the desired signal with single ended amplifiers (as are often used in currently available biomedical instruments and as shown in FIG. 2), and even relatively small noise signals (e.g. a few mVs), may still tremendously affect recording of weak physiological or neural response signals at their uV level.

Introduction of external noise is almost inevitable because of the physical connection between the signal source (i.e. the patient or test subject 30) and the noise sources (the environment, the computer interface, the power supply, etc.). Although isolation amplifiers (e.g., 32) are always used in physiological signal amplification, AC noise can still be coupled to the amplifier input and output, causing superposition of the noise. This noise becomes an obvious problem when the weak signals are of interest and noise level is especially high.

Automatic Adjustment of DC Offset and Gain:

Turning now to automatic Adjustment of DC Offset, for a conventional DC amplifier, DC tolerance depends on the gain and the voltage of the power supply. For example, if the gain is a multiplier of one (×1), and the voltage of power supply is plus or minus (+/−) five volts (5V), then the amplifier's DC tolerance is near +/−5V. If the gain is increased to ×100, the DC tolerance is then +/−5V/100=+/−50 mV. For a conventional DC amplifier with output of 0.5V and input of 5.0 uV, its gain is ×100K and the DC tolerance is 50 uV. This is not acceptable for any amplifier with sensitivity of 5.0 uV, not to mention that of 0.5 uV, because such a DC amplifier would be practically saturated all time long unless there is guarantee that the DC offset of the input signals is always smaller than 5.0 uV.

Larger DC tolerance (e.g., greater than one volt) is preferred but not always necessary. As the electrode interface potential is no more than 200 mV, and the DC component of physiological signals can be correctly measured when it is amplified close to +/−0.5V, the DC tolerance of +/−1.0V for a DC amplifier is reasonably acceptable in most cases. Furthermore, since the measurement accuracy of 5% for most physiological signals is considered as good fidelity, a measure error tolerance of 1% or 10 mV for a DC amplifier is also acceptable.

In accordance with the present invention, wearable, digital DC amplifier 58 provides a sensitivity of 0.5 uV as well as DC tolerance of +/−5V, in part, by employing a 24 bit (19 bit effective data) A/D converter 78 to provide a sensitivity of +/−2.5V/262144=0.0000095V=+/−9.5 uV, and a DC tolerance of +/−2.5V. An analog DC amplifier with a gain ×100 further amplifies the physiological signals for 0.1 uV sensitivity. Since such an amplifier tolerates signals up to +/−2.5V/100=25 mV DC, digital potentiometer 86 is used to adjust the DC offset at the amplifier input 70. The idea is to offset or subtract the neural response signal's DC component from the amplifier input so that the net input has a DC component no larger than 25 mV. The DC offset adjustment is expected to be required only once and does not need to be repeated because the DC component does not change quickly with time.

For an 8 bit digital potentiometer 86, the accuracy of the DC output is +/−1.0V/128=+/−7.8 mV. Therefore, when a voltage with such accuracy is used to subtract the DC offset at the ×100 amplifier input, the possible net DC offset is no larger than +/−7.8 mV, and the amplifier output is +/−7.8 mV×100=780 mV. This configuration provides +/−1.0V DC tolerance and add 8 bits of dynamic range.

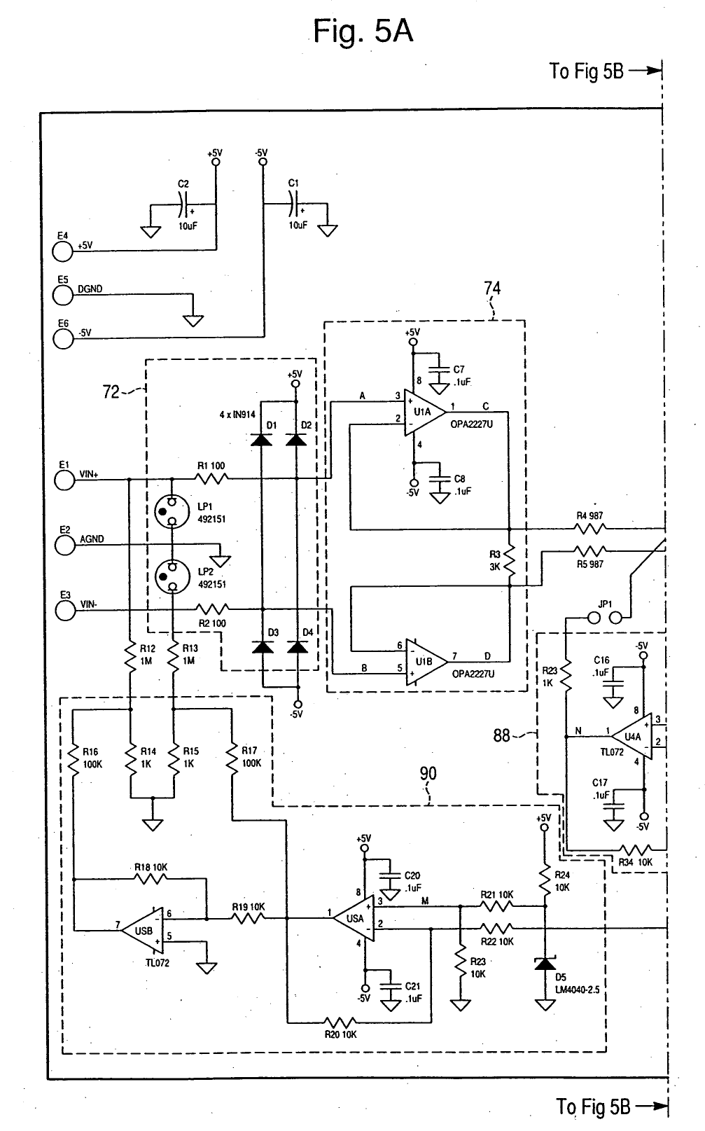

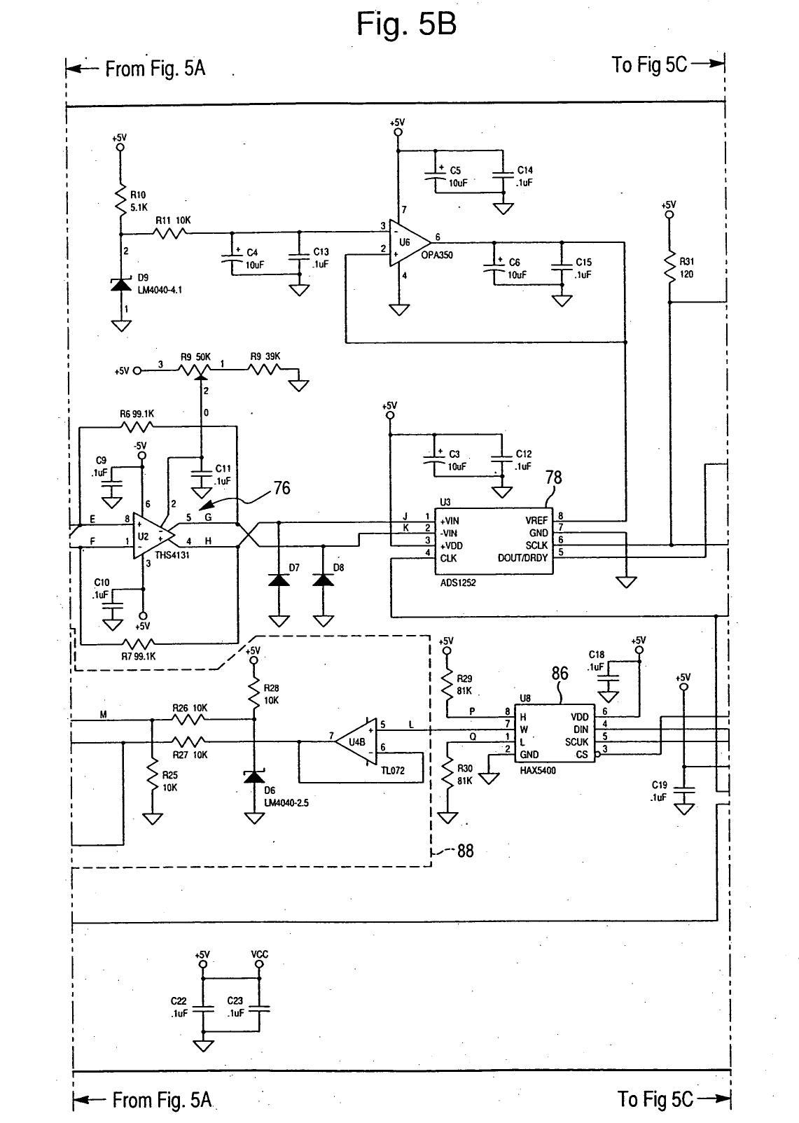

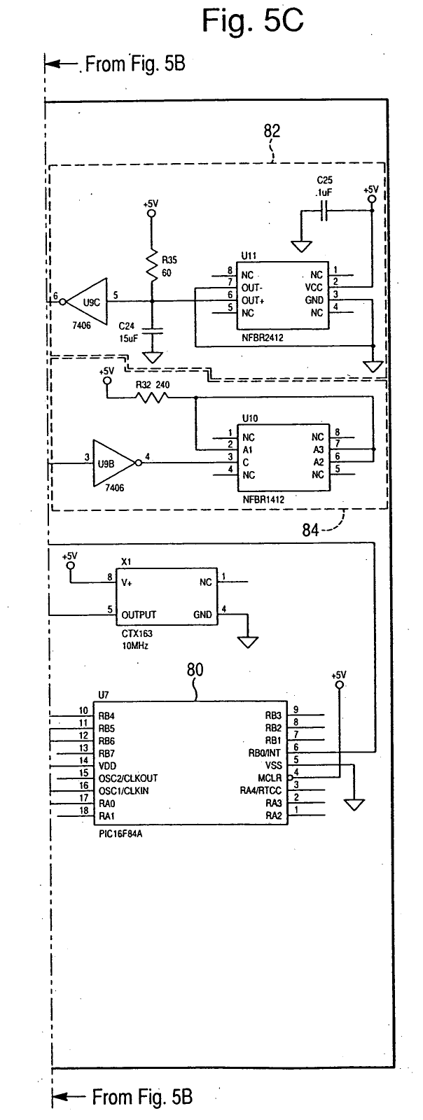

Once the DC component is adjusted, the dynamic range for AC components uses the remainder of the input tolerance, which is 100 mV-78 mV=22 mV. In other words, the configuration of FIG. 4 (and FIGS. 5a-5c) roughly gives a +/−1.0V pure DC tolerance with 78 mV measure error tolerance and +/−22 mV to 1.0V pure AC dynamic range with 0.1 uV signal sensitivity.

The adjustment of digital potentiometer 86 is based on the feedback of ADC data through ADC 78. The program for computing offset is stored in memory resident on the PC board with microprocessor 80 and is illustrated in flow diagrams 9c and 9d. For an 8 bit digital potentiometer, the procedure takes 8 iterating steps by using the progressive approximation method. This procedure usually takes less than a millisecond. The features of large (+/−1.0V) dynamic AC/DC range for minimum 0.1 uV sensitivity and continuous frequency band near DC allow the user to record DC or near DC signals, such as c-wave in ERG, slow waves in EEG, and slow components in ERP without sacrificing any slow AC components.

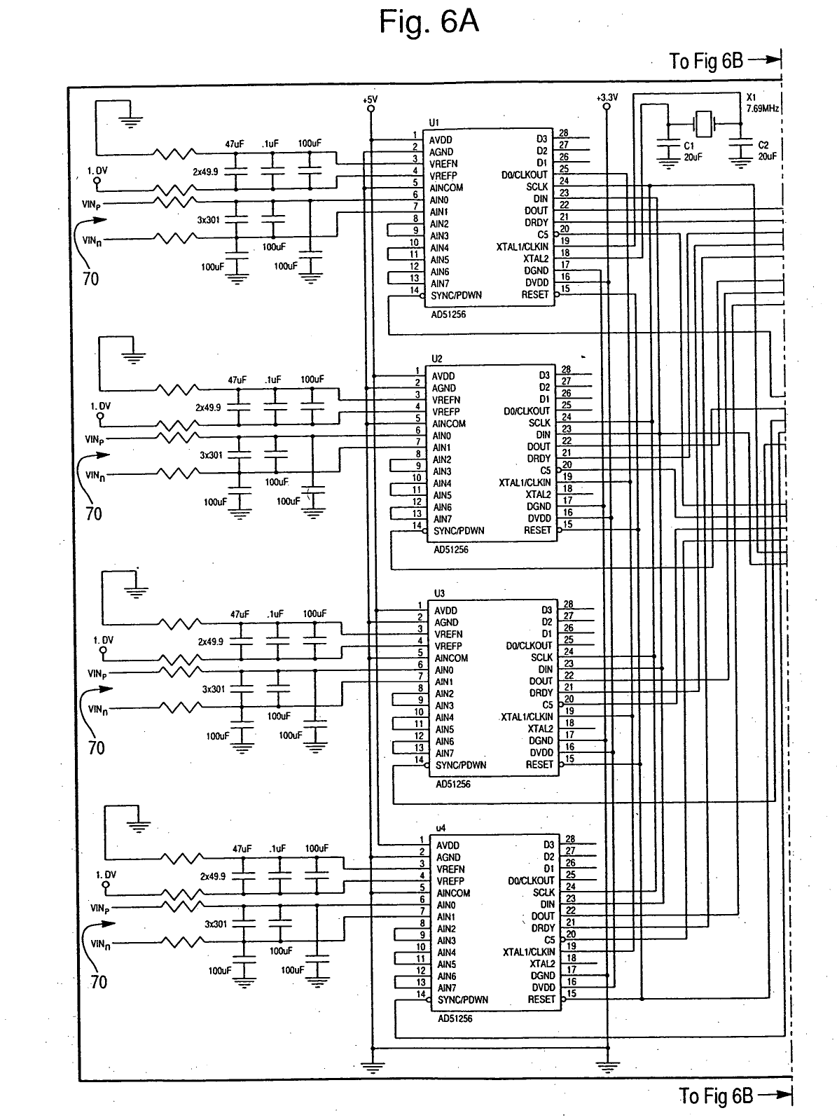

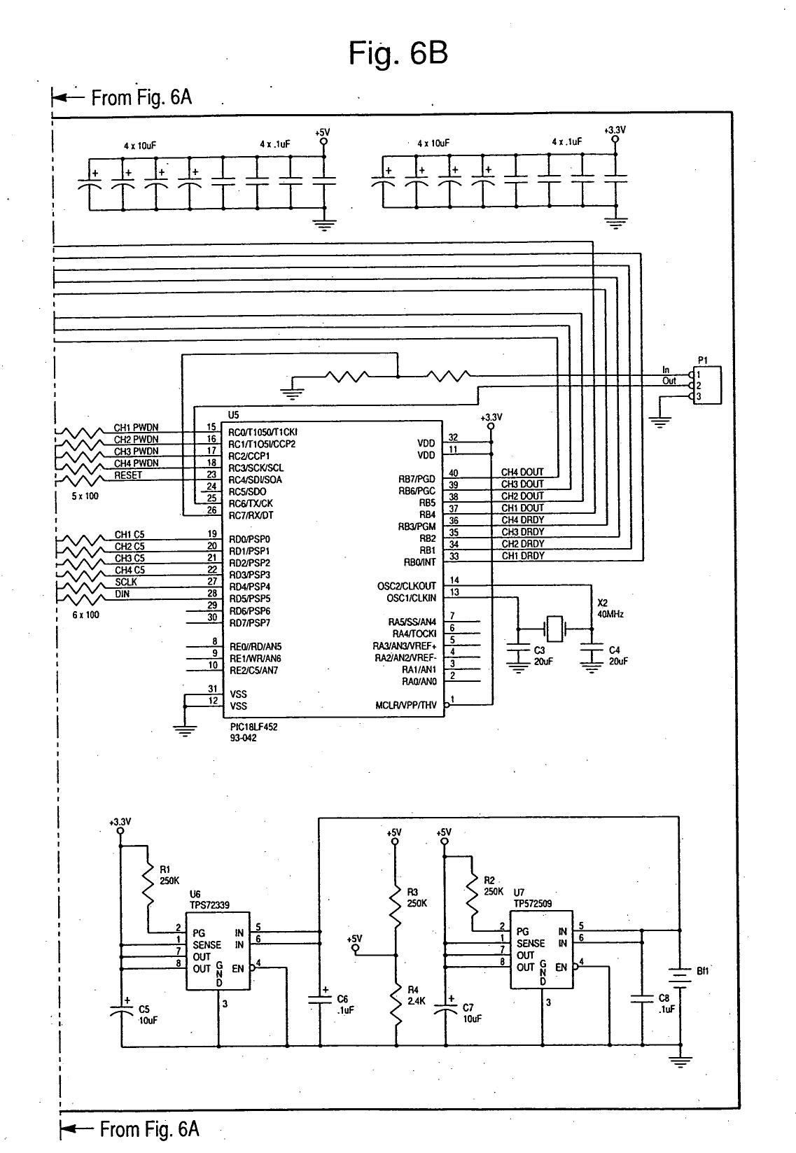

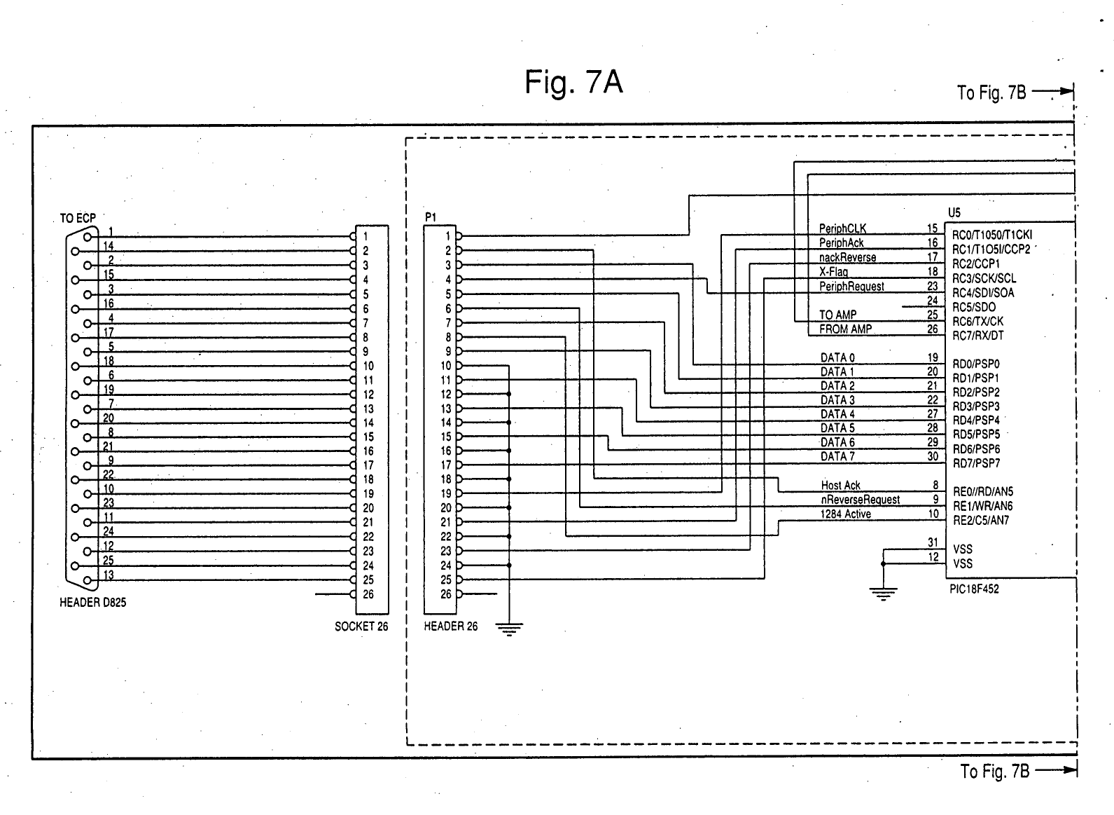

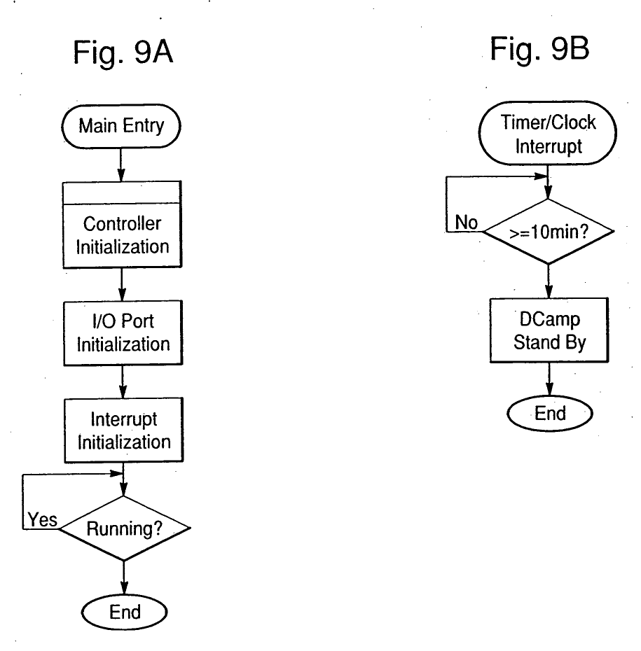

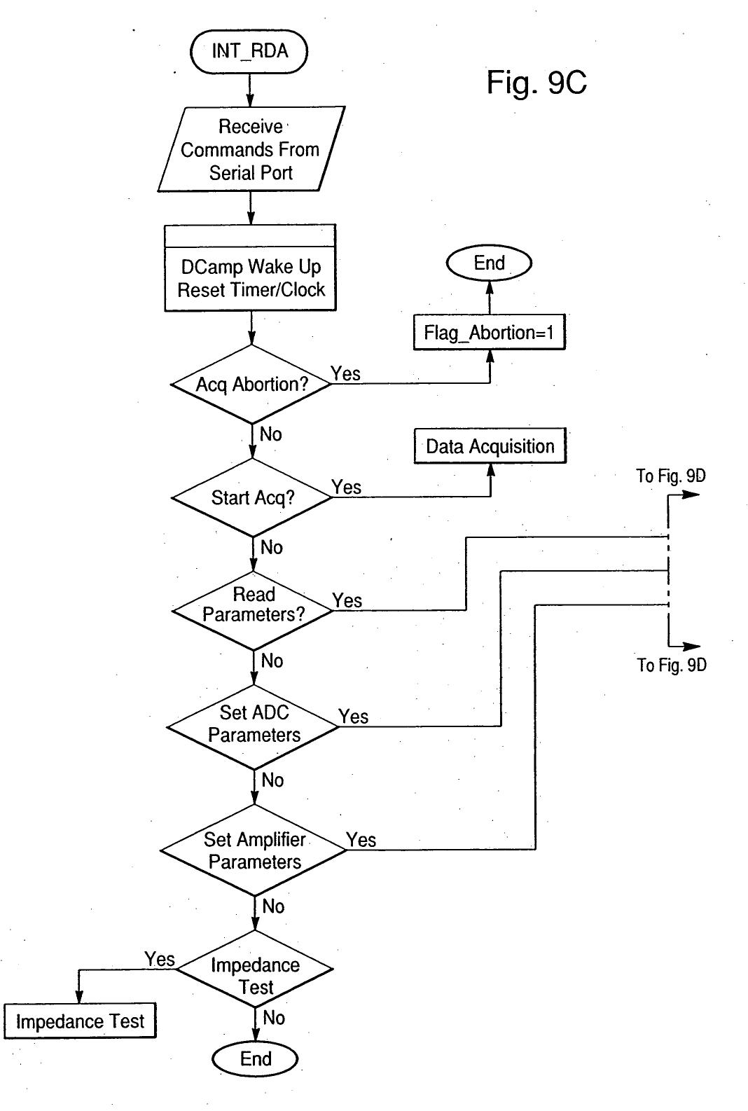

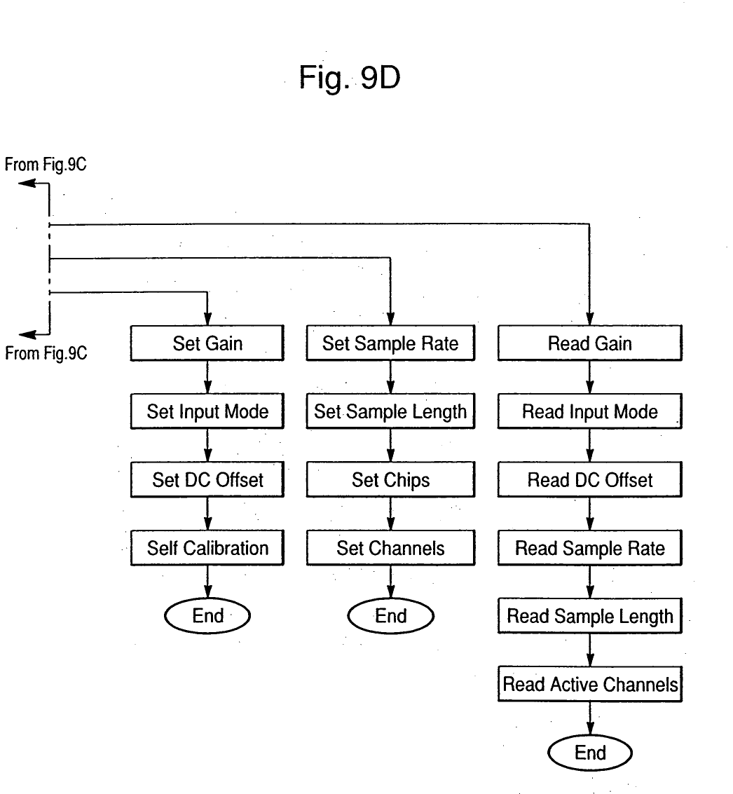

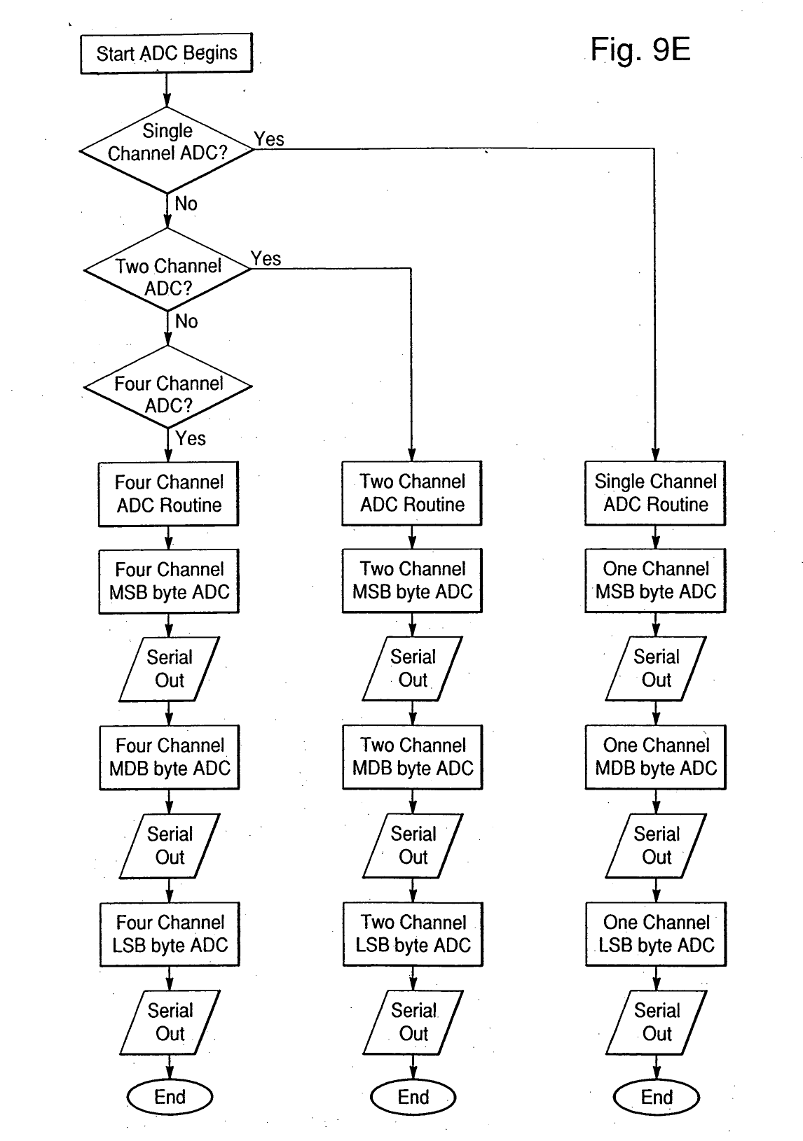

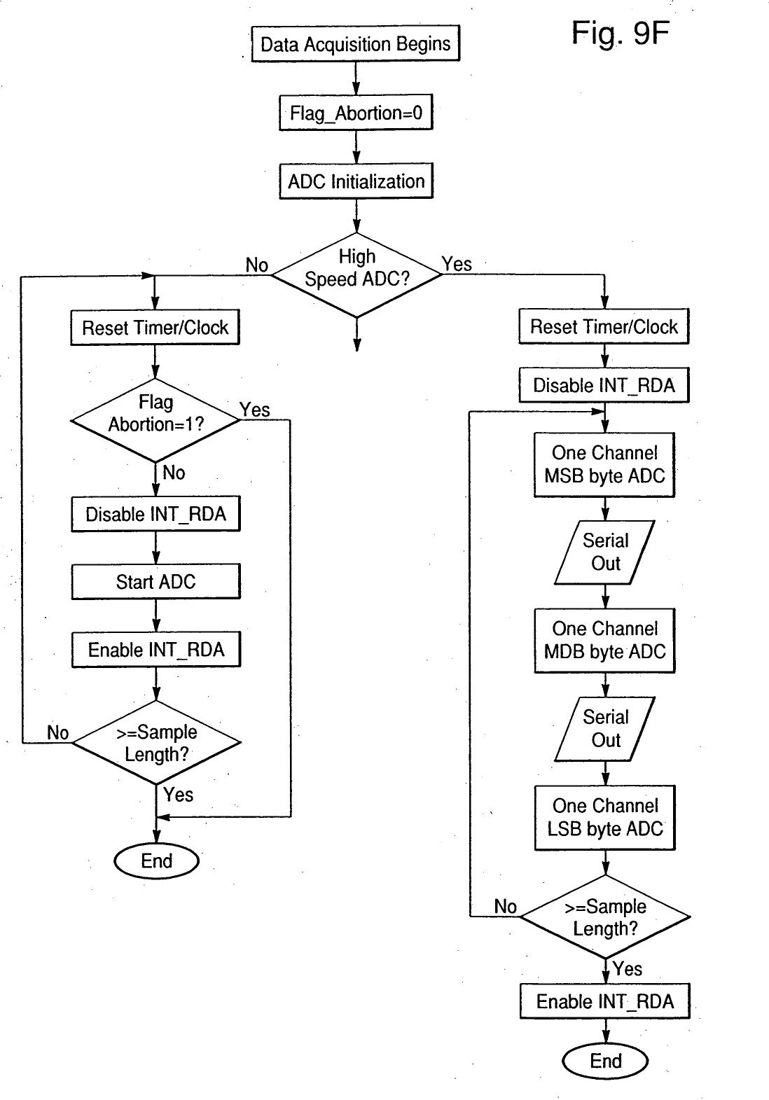

FIGS. 9a through 9f are a process flow diagram illustrating pre-programmed method steps executed using the microprocessor in the wearable DC amplifier unit 58, various embodiments of which are illustrated in FIG. 4, FIGS. 5a-5c and FIGS. 6a and 6b. Some signals, such as ECG, EOG, and BP signals, are relatively large (in mV or above). There's no need a pre-amplifier stage, and so no DC offset adjustment. The sensitivity for patient amplifier unit 58 is about +/−1 uV with +/−1.0V DC tolerance. Some signals, such as ERG and VEP signals (in uV or above), may need a low gain (×2 to ×5) to achieve the best SNR results. Some extremely weak signals, such as focal-ERG and miniature end-plate potentials (less than 1 uV), need a gain from ×10 to ×100 to overcome the noise limitation. Therefore, programmable gain amplifier stage 76 is responsive to a pre-programmed control algorithm stored in memory (and shown in the flow diagrams of FIGS. 9a-9e) for use in microprocessor 80 for more flexibility and use in a wide variety of applications.

Electrically Isolated Communication Between Amplifier Unit and Interface Unit

Neural Response signals are amplified and converted to digital form in the amplifier unit 58 before transfer via the controller interface 62 to the user's computer 64. The gain adjustable pre-amp stage 76 and the A/D converter 78 are also responsive to and controlled by the user's computer 64 through the interface unit 62. A bidirectional fiber optic cable 60 provides a data communication pathway to eliminate the possibility of introducing external electromagnetic interference or noise to the patient amplifier and ensures the patient's safety from exposure to any possible current leakage from the external power supply. A power supply is typically connected to high voltage power line electrically via a transformer. The fiber optic link 60 provides the most reliable, secure, and safest data communication method compared to other methods such as RF, IR, and sonic communications as well as the conventional electrical wire connections. When patient safety is not a priority in the signal amplification (e.g., as for animal experiments) amplifier unit 58 may be connected directly to interface controller 62 without fiber optics. Since the data transfer rate can reach 90 kilobytes/sec (30 ksps for 3 bytes per samples), a moderately high speed data communication device is needed.

Power Supply for the Patient Amplifier:

External power supplies using an AC power outlet give rise to safety issues. Currently available patient amplifiers always utilize an isolation transformer to float power supply voltage separate the external power line for the patient's safety. The isolation transformer is a major noise source, both electrically and magnetically and since all related devices including the patient amplifier, computer interface, stimulation device, computer monitor, printer, etc. are electrically connected, all the devices must be electrically isolated and so are powered through the isolation transformer. This results in a requirement for a costly, large and bulky transformer. In a preferred embodiment, as shown in FIG. 6b, a rechargeable battery is provided to power patient amplifier unit 58 to minimize noise introduction and to maximize patient safety.

Because amplifier 58 uses fiber optics for data communication, amplifier 58 is completely electrically isolated from all other electrical devices. During the battery recharging time, patient amplifier unit 58 is disconnected internally by relays (as shown in FIG. 6b), and so patient safety is ensured and noise is reduced. A single charge of the battery should last for the duration of a normal shift (e.g., about 8 hours) or longer, especially if a spare removable battery pack unit is available for use in amplifier 58.

Current consumption is the major concern when battery power supply is applied, as it needs to last at least the working hours. At present, the rechargeable battery with the highest capacity is the lithium-ion battery, which can reach 1200 mAh at the portable size of 15×20×40 mm and weight of 50 g. Another choice is to use NiHM battery, which also has high capacity and no memory effect. The pitfall is that it continually discharges, even when not in use (typically losing charge at 3% per day).

Interface Controller Unit, ECP/USB Controller, and its Driver

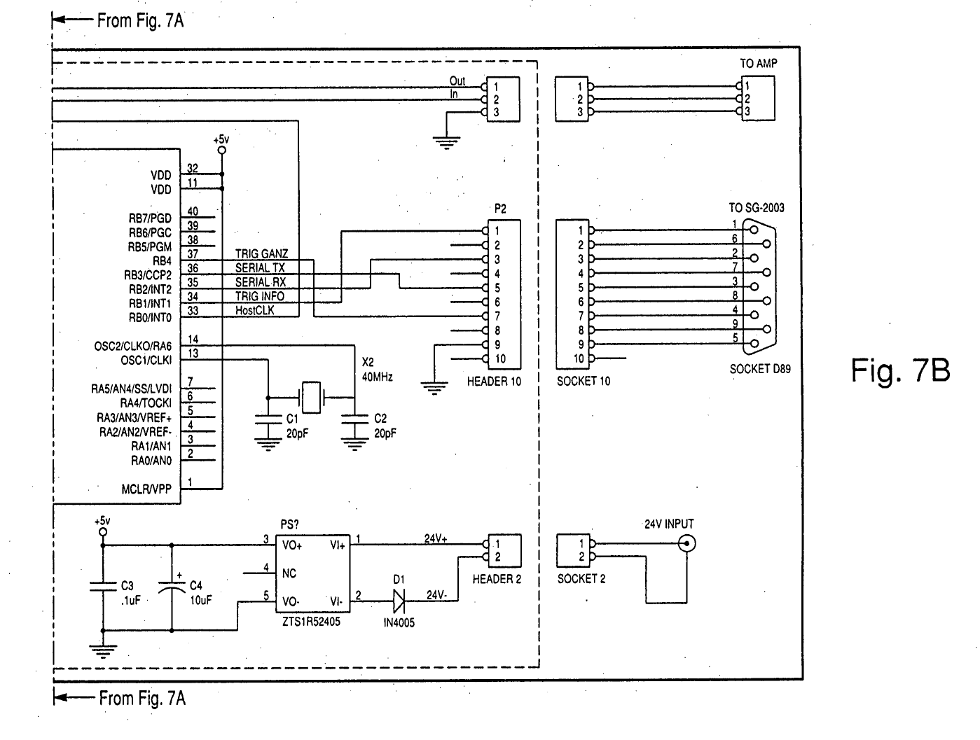

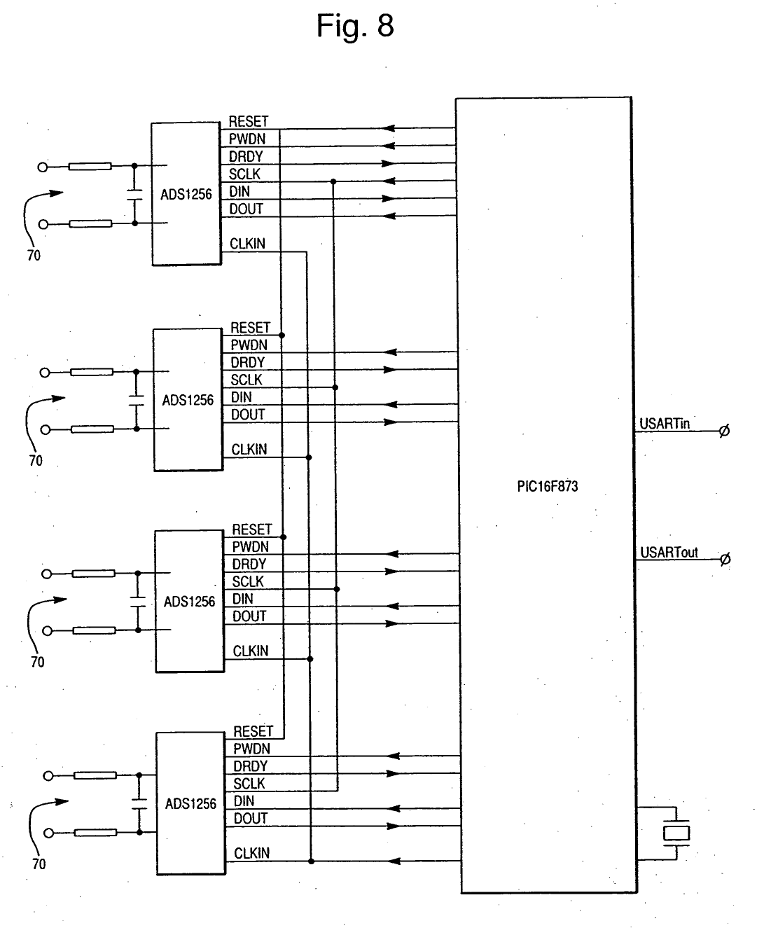

FIGS. 7a and 7b are schematic diagrams of circuitry included in interface control 62 for use in the system of FIG. 3, in accordance with the present invention. The patient amplifier unit 58 is controlled by the interface unit 62 via an optically isolated fiber optics communication link 60. The acquired neural response signal data is then fed to the user's computer 64 through an ECP/USB connection 66. In a preferred embodiment, interface unit 62 provides extra digital I/O and TTL compatible trigger I/O ports. In many cases, especially in the data transfer and synchronous stimulation device trigger in/out modes, the operations are time critical (requiring less than 1 msec of time jitter), and so a dedicated high speed processor is preferable for completing the tasks.

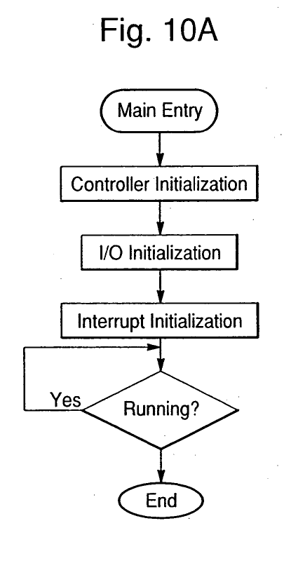

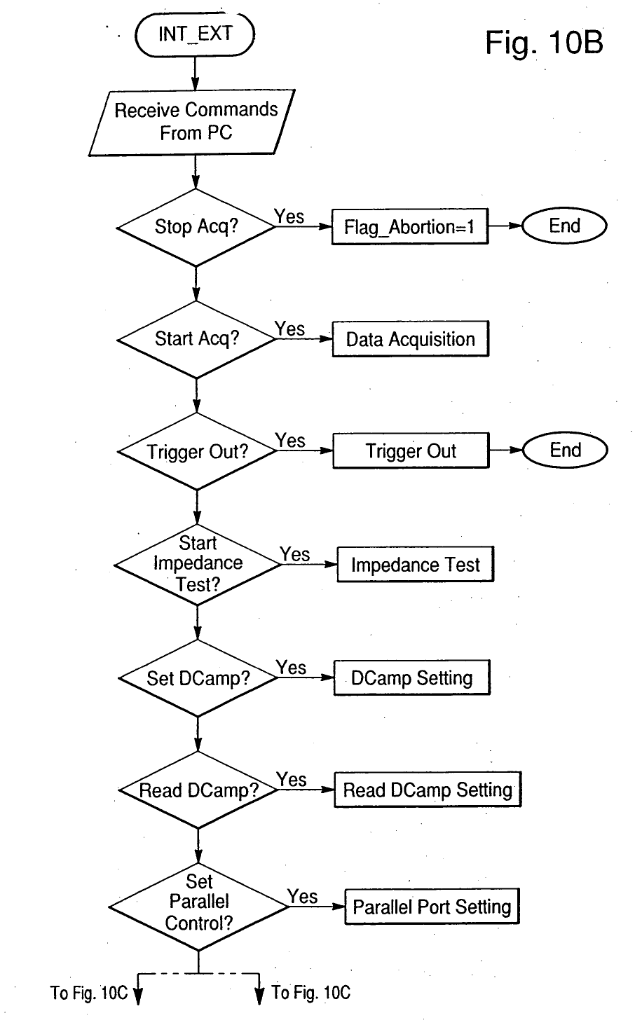

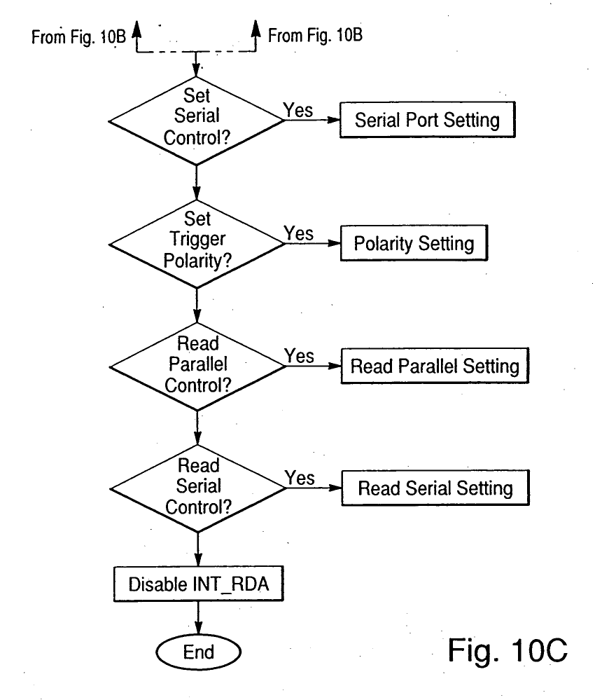

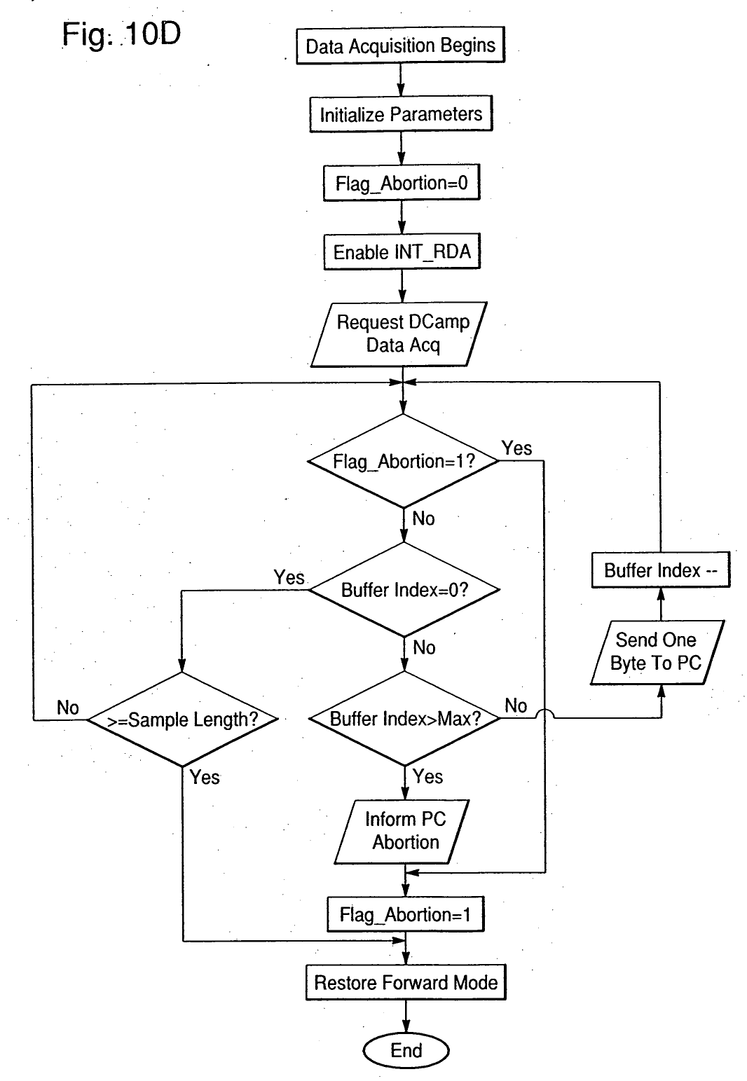

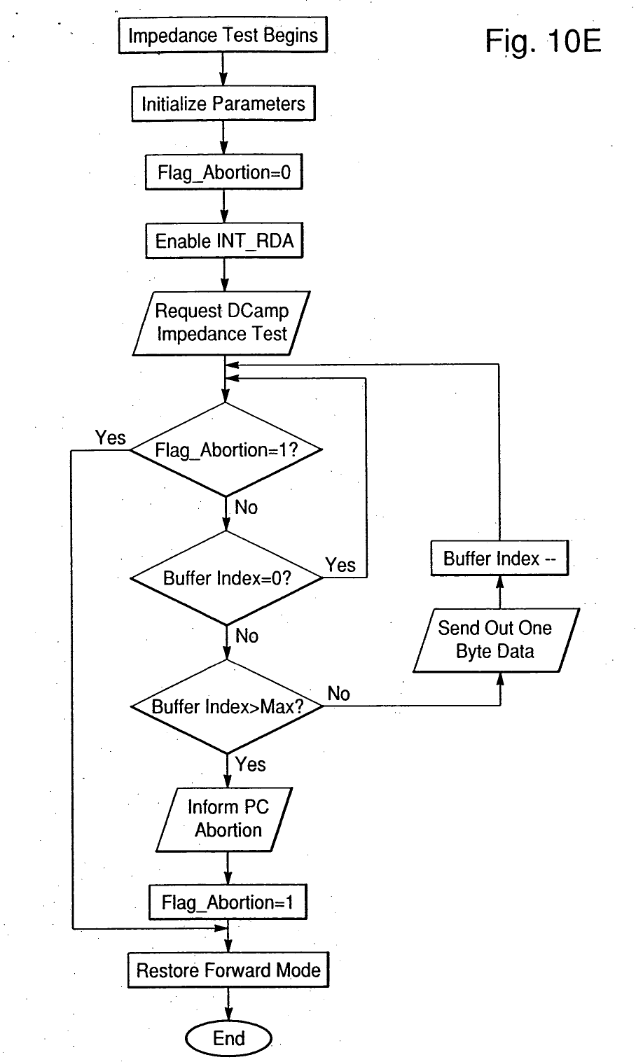

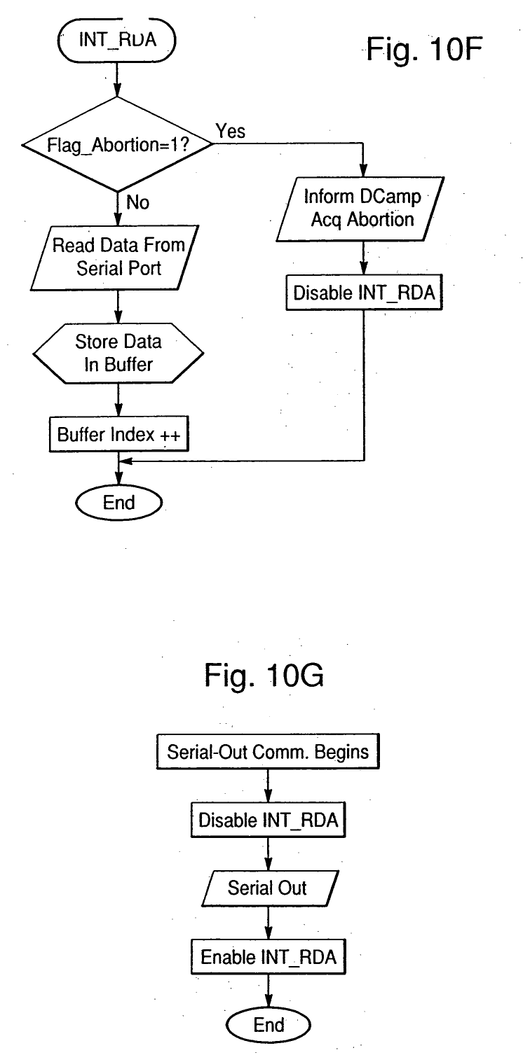

FIGS. 10a through 10g are a process flow diagram illustrating pre-programmed method steps executed by the interface controller 62 when used to communicate with and control the wearable DC amplifier unit 58.

The interface unit 62 also serves as a data buffer when communicating with the host computer 64. For many operating systems (e.g., Microsoft 98/ME/NT/XP) the guaranteed highest time resolution is 1 msec and so the data need to be buffered before transmission in packets. Since the data transfer rate is high at its highest sampling rate (30 kHz), (and up to 90 kilobytes/sec per channel) the data buffer should be at least 90 bytes or above per channel. Interface unit 62 includes a high speed microcontroller, which interprets the requests from the computer or digital I/Os and, as shown in the flow diagrams of FIGS. 10a-10g, executes the following tasks:
Select the number of active channels;
Set parameters for the DC amplifier unit;
Reset the DC offset and measure the DC component;
Start an ADC sweep at the given sample rate and length;
Buffer the acquired data and transfer to host computer;
Set the digital I/O parameters; Set parameters for the stimulation devices;
Transceiver commands and settings between peripheral stimulation devices and host computer;
Perform preliminary digital signal processing.

The USB computer interface connection 66 is preferable due to its ease of use, high speed, reliability, flexibility, and low power consumption. Currently, the most available high speed computer interface port is the USB 1.1 port, which supports up to 115 peripheral devices at the speed up to 12 Mbytes/sec.

USB has 3 data transfer modes: xxx. The data packet is the basic unit where transferred data are packed into. Each packet can hold up to 1024 bytes and transferred at minimum 1 msec intervals. Although it is convenient for a user to plug in the USB interface unit and start using it, the challenge of designing such an interface is that the data communication through USB is complicated and needs to be controlled by a USB controller on the peripheral side.

Cypress Inc. provides a series of USB controllers which integrate CPU, I/O, and USB ports. For example, the SL811HS is a USB tranceiver with built in 240 bytes FIFO buffer. Cypress Inc. provides a complete development kit package for both controller and Microsoft Windows 98/Me/XP USB driver.

Specifications for the Wearable DC Amplifier:

Wearable DC amplifier 58 preferably has the following features:

1. Ultra-low noise differential amplifier in order to reach the goal of ultra-low noise (<1.0 uV RMS) with full differential amplification (differential input and differential output).

2. Battery power supply to further increase safety, lower noise, and improve the performance. The battery is rechargeable, and lasts at least 8 hours for one charge.

3. Sample rate of up to 30K samples per second (sps); preferably sample rate is program selectable for a plurality of rates, e.g., 15K, 7.5K, 2K, 1K, 500, 200, 100, 60, 50, 30, 25, 10, 5, 2.5, and 1 sps. The DC to 15 kHz bandwidth (30,000 sps ADC), which meets requirements for measuring a variety of different physiological signals, including, for example, EEG, EMG, ECG, ERG, VEP, ERP, BP, AP, membrane potential, single channel recording, smooth muscle activities, optical recordings, and others. It is expected that all biomedical signals are applicable for this amplifier.

4. Resolution of up to 24 bits (21 bits effective data at 2K sps) to cover the dynamic range from 1.0 uV to 1.0V without amplification gain in the pre-amp stage 76. The ADC resolution can go up to 27 bits at 2K sps with addition of an 8 bit digital potentiometer (e.g., 86) to adjust any possible DC offset and so allow at least +/−1.0 DC tolerance. The accuracy of DC offset adjustment is <10 mV and so the amplification gain can be set to ×2, ×4, ×8, ×16, ×32, and ×64 in the pre-amp stage 76.

5. Direct digital output from the amplifier unit avoids further A/D conversion with possible noise addition.

6. Fiber optic connection 60 between the patient amplifier unit 58 and the computer interface unit 62 eliminates pickup of external noise and enhances resistance to external disturbance and interference.

7. USB connection 66 between the interface unit 62 and host computer 64 for flexible plug-n'-play connectivity and high speed data communication.

8. Interface unit 62 controls amplifier unit 58, receives the acquired data and transfers the buffered data to host computer 64 upon request. Interface unit 62 also controls the optional stimulation device 40 (e.g., ganzfeld stimulator or pattern monitor). For more general purpose applications, a high speed (up to 1 Mbytes/sec) parallel port and a low speed (9600 baud rate) serial port are implemented in interface unit 62.

9. The synchronous signal lines are connected to the interface unit's Trigger-In and Trigger-Out ports, with <10 uS response accuracy.

10. Two approaches are used to achieve multi-input channel capability: use the duplicated ADC circuits and the multi-channel ADC chips. Preferably, a four ADC chip configuration ensures simultaneous data acquisition for up to four channels at the highest sample rate. For lower sample rates, four ADS1256 chips support up to 32 single ended channels.

11. Optionally, when the amplifier is used in animal/cell studies where electrical safety is not an issue, the battery power supply and fiber optics might be replaced with a standard AC power supply and a copper wire data cable.

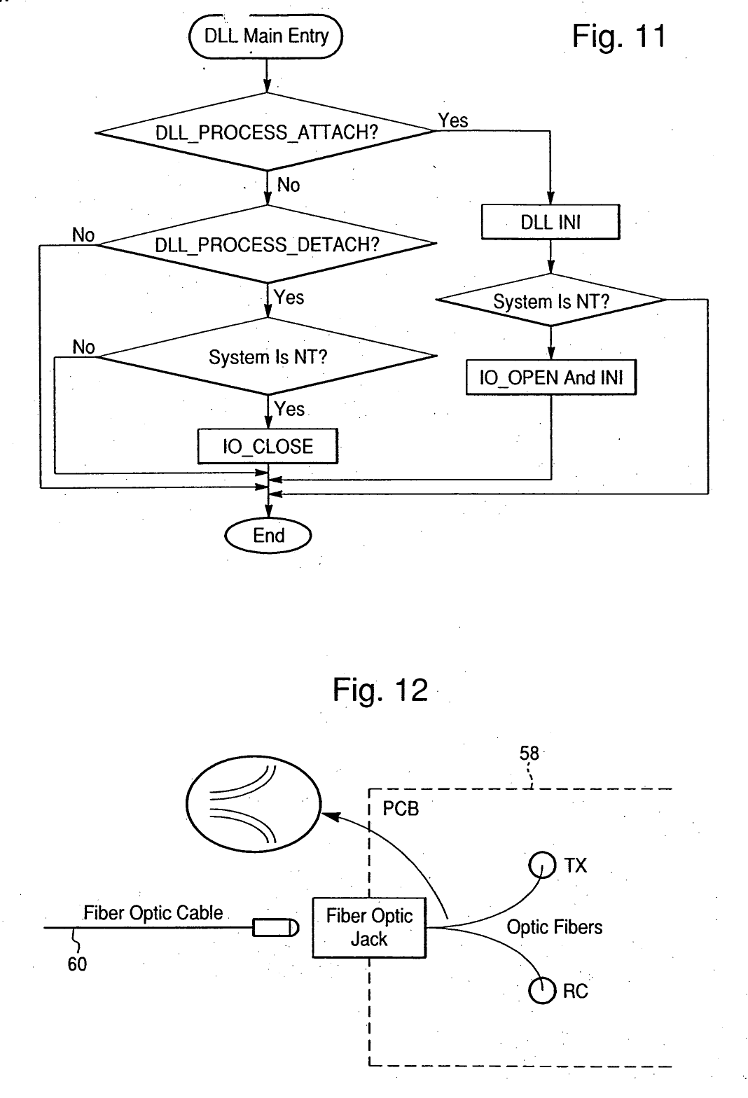

12. Optionally, some basic digital signal processing routines (such as data binning, high-cut filtering, low-cut filtering, notch filtering, etc., see below) are pre-programmed in the host computer 64. This will allow user's calls for signal processing (see the flow diagram of FIG. 11).

Applications for the DC Amplifier:

In use, DC amplifier 58 is designed for all biomedical signal recordings with the highest safety, the greatest dynamic range, the best data quality, and the most flexible convenience considered. Intended applications include:
Conventional ERG, multi-focal ERG, focal ERG, and dc-ERG recordings, where uV signals in near DC to 500 Hz with mV DC offset are of interest;
Conventional EOG and fast EOG recordings, where sub-mV to mV signals in 0.1 to 100 Hz are of interest;
EEG, VEP, and ERP recordings, where uV signals in near DC to 100 Hz with mV DC offset are of interest;
ECG recordings, where mV signals in near DC to 100 Hz with mV DC offset are off interest; EMG recordings, where uV to mV signals in 1.0 to 1000 Hz are of interest;
Action potential, membrane potential, synaptic potential, miniature endplate potential, patch-clamping, and extra-cellular/intra-cellular potential recordings, where uV to mV signals in DC to 2000 Hz are of interest;
Blood Pressure, pulse, respiration, heart beat, flow rate, blood glucose, pH value, ion concentration, etc., by using corresponding transducers, where mV to V signals in DC to 10 Hz are of interest;

Optical recording/imaging using photodiode arrays for measuring membrane potential, intra-/extra-cellular ions (e.g. Ca++, K+, Na+, H+), and molecules, where uV signals in DC to 500 Hz with up to volts DC offset are of interest.

TABLE 1
DC amplifier 58 specifications:

| | |
|---|---|
| Analog Input: | differential/single-ended |
| N. Channels: | 4 (differential) high speed, 32 (single-ended) |
| | low speed |
| Sensitivity: | <1 uV (Johnson/shot noise <0.5 uV RMS, |
| | digital bit noise <0.05 uV) |
| Analog Input | 80 Mohms |
| Impedance: | |
| Frequency range: | DC - 15 kHz (AC is sample rate dependent) |
| ADC Sample Rate: | up to 30 K sps |
| ADC resolution: | 24 bits |
| DC tolerance: | no less than +/−1.0 V |
| Filters: | Low-cut, High-cut, & Notch (digital), |
| Output Format: | Digital USB 1.1 |
| Electrical isolation: | Fiber optics |
| Power consumption: | <100 mA (peak) |
| Power supply: | Rechargeable Battery for >8 hours continuous use |

Command List for Communicating with the DC Amplifier Interface:

The command list for the DC amplifier ECP interface 62 is for the Microchip microprocessor PIC18F452 that controls the communication between the DC amplifier 58 and the ECP port connected to the user's PC 64. The first byte (Byte0) is the command sent from the computer to the interface unit. Some commands are followed by one or more byte(s) to be sent to the interface unit. This can be up to 4 bytes, as used in the setting channel command. Some commands expect receiving data from the interface unit. For example, ReadSingleADC command would expect 4 bytes or more coming from the interface unit, depending on the number of channels. ReadBatchADC command would get more bytes, DataLength×4×NumChan in total.

TABLE 2
Interface Commands

| Command | | | | | |
|---|---|---|---|---|---|
| Return | Byte0 | Byte1 | Byte2 | Byte3 | Byte4 |
| ResetInterface | 0x01 | | | | |
| ResetADC | 0x02 | | | | |
| SetChannel | 0x03 | FlagChan0 | FlagChan1 | FlagChan2 | FlagChan3 |
| SetInputMode | 0x04 | 0 or 1 (0 = diff, 1 = single-ended | | | |
| SetDataLength | 0x05 | Len_Hi | Len_Low | | |
| ReadDataLength | 0x06 | | | | |
| SetSampleRate | 0x07 | Rate_Hi | Rate_Low | | |
| ReadDataInterval | 0x08 | (>=50 in usec, <50 in Hz) | | | |
| 2bytes | | | | | |
| ImpedanceOnOff | 0x09 | Current (100, 5, 2, 0 uA/10) | | | |
| BufferOnOff | 0x0A | 1 or 0 (1 = on, 0 = off) | | | |
| SetGain | 0x0B | chip 1, 2, 3, 4 | 1, 2, 4, 8, 16, 32, 64 | | |
| OffsetCalibration | 0x0C | | | | |
| GainCalibration | 0x0D | | | | |
| AutoCalibration | 0x0E | | | | |
| ReadSingleADCall | 0x10 | | | NumChan × 3 | |
| ReadSingleADCch. | 0x11 | NumChan | | | |
| 3bytes | | | | | |
| ReadBatchADCall | 0x12 | | | Num × | len × 3 |
| ReadBatchADCch. | 0x13 | NumChan | | | len × 3 |
| StopReadADC | 0x14 | | | | |
| CheckTrigIn | 0x15 | | | | |
| yes/no | | | | | |
| SendTrigOut | 0x16 | | | | |

| | | |
|---|---|---|
| SendSerialOut | 0x20 | number of bytes? |

| | | |
|---|---|---|
| ReadSerialIn | 0x21 | num. of bytes? |
| SendParalOut | 0x22 | |
| ReadParalIn | 0x23 | |
| ReadSerialNumberAmp | 0x2E | |
| ReadSerialNumberInterface | 0x2F | |

TABLE 3
Interface Channel Flags

| Flag Channel On/Off | FlagChan0 | FlagChan1 | FlagChan2 | FlagChan3 |
|---|---|---|---|---|
| 0 = off, 1 = on | ch. 0-7 | ch. 8-15 | ch. 16-23 | ch. 24-31 |

TABLE 4
DC Amp Sample Rates

| ADS1256 Sample Rate | Application Use Rate | Rate_Hi | Rate_Low |
|---|---|---|---|
| 30000 | 20000 | 0x4E | 0x20 |
| 15000 | 10000 | 0x27 | 0x10 |
| 7500 | 5000 | 0x13 | 0x88 |
| 3750 | 2500 | 0x09 | 0xC4 |
| 2000 | 2000 | 0x07 | 0xD0 |
| 1000 | 1000 | 0x03 | 0xE8 |
| 500 | 500 | 0x01 | 0xF4 |
| 100 | 100 | 0x0 | 0x64 |
| 60 | 60 | 0x0 | 0x3C |
| 50 | 50 | 0x0 | 0x32 |
| 30 | 30 | 0x0 | 0x1E |
| 25 | 25 | 0x0 | 0x19 |
| 15 | 15 | 0x0 | 0x0F |
| 10 | 10 | 0x0 | 0x0A |
| 5 | 5 | 0x0 | 0x05 |
| 2.5 | 2 | 0x0 | 0x02 |

TABLE 5
Interface Word Lengths

| Data Length | Len_Hi | Len_Low |
|---|---|---|
| infinite length | 0 | 0 |
| 256 | 01 | 0 |
| 512 | 02 | 0 |
| 1024 | 04 | 0 |
| 2048 | 08 | 0 |
| 4096 | 10 | 0 |
| 8192 | 20 | 0 |
| 16384 | 40 | 0 |
| 32768 | 80 | 0 |
| 65536 | FF | FF |
| Any length (0-65535) | xx | xx |

Since amplifier 58 may lock up the control interface 62, interface status should be checked every time before commands are sent. The value of amplifier status reflects whether the amplifier is working normally. Table 6 sets forth the possible situations:

TABLE 6
Amp/Interface Status Commands

| Value | Meaning |
|---|---|
| 0 | Working normally |
| 1 | Interface unit power not on (no response) |
| 2 | Amplifier power too low |
| 3 | Amplifier not connected or power not on |

All the data is internally converted to uV in float by the driver, and so the user does not need further scale unit conversion. The following sets forth a function list for the class communicating with the interface unit:

TABLE 7
Interface Data communication functions

```
  // 1. To reset the amplifier and interface:
bool AmplifierReset( );
bool InterfaceReset( );
  // 2. To obtain a single AD data:
bool AnalogIn(float &dataAddr);
  // 3. To request a batch of AD conversion:
bool AnalogIn(long &rate, long &length, float *dataAddr);
bool AnalogIn(long &rate, long &length, float *dataAddr, int chan);
  // 4. To request a batch of AD conversion with trigger out:
bool AnalogInBatchTrigger(long &rate, long &length, float *dataAddr);
bool AnalogInBatchTrigger(long &rate, long &length, float *dataAddr,
int chan);
  // 6. To send a single digital command via the interface unit serial port:
bool SerialOut(UCHAR command);
  // 7. To request a single digital data via the interface unit serial port:
bool SerialIn (UCHAR &data);
  // 8. To send a single digital command via the interface unit serial port:
bool ParalOut(UCHAR command);
  // 9. To request a single digital data via the interface unit serial port:
bool ParalIn (UCHAR &data);
  // 10. To get the amplifier and ADC status
bool GetAmpStatus(UCHAR &status);
bool GetCurrentADLength(int &length);
  // 11. To set periodic trigger out in msec
bool SetTriggerOutPeriodic(int interval);
  // 12. To Start periodic trigger out
bool StartTriggerOutPeriodic( );
  // 13. To stop periodic trigger out
bool StopTriggerOutPeriodic( );
  // 14. To check trigger in
bool CheckTriggerIn(BOOL in);
  // 15. To set trigger in call back
bool CallBackTriggerIn(void *CallBackAddress);
  // 16. To send a trigger out signal
bool SendTriggerOut( );
  // 17. To put the amplifier in standby mode and wake it up
bool PowerSaveMode( );
bool WakeupMode( );
  // Successful if the function returns 0, otherwise error (need an error
message list).
```

TABLE 8
Interface Parameter setting functions

```
  // 1. To set sampling rate
int SetSampleRate(int rate); // return actual sample rate, not valid value if
zero returned
int GetSampleRate( );
  // 2. To set data length
int SetDataLength(int length); // return actual length
int GetDataLength( );
  // 3. To set channel
void EnableADFlag(UCHAR *FlagChan, bool enable);
void EnableADCChan(int channel, bool enable);
bool GetEnabledChannels(UCHAR *FlagChan);
  // 4. To set low-cut frequency
void SetLowCutFrequency(float lowCut);
  // 5. To set hi-cut frequency
void SetHiCutFrequency(float hiCut);
  // 6. To get low-cut frequency
float GetLowCutFrequency( );
  // 7. To get hi-cut frequency
float GetHiCutFrequency( );
  // 8. To set input mode
void SetAmplifierMode(bool SingleEnded);
bool GetAmplifierMode( );
  // 9. To set amplifier gain
void SetAmplifierGain(UCHAR chip, UCHAR gain);
UCHAR GetAmplifierGain(UCHAR chip);
  // 10. Calibrations
void CalAmpGain( );
void CalAmpOffset( );
void CalAmpSystem( );
  // 11. To set trigger signal polarity
bool SetTriggerInPolarity(BOOL pos);
bool SetTriggerOutPolarity(BOOL pos);
  // 12. To set online data processing feature
bool SetOnLineNotch(int frequency, BOOL on); // default is off
bool SetOnLineBinning(int binWidth); // default is 0. This will affect Data
Rate!
bool SetOnLineSmooth(int bin); // default is 0;
```

TABLE 9
Interface Data processing functions

```
  // 1. Low-cut filtering
void LowCutFilter(float *data, int length, float freq, float DataRate, int
type);
  // 2. Hi-cut filtering
void HiCutFilter(float *data, int length, float freq, float DataRate, int
type);
  // 3. Band width filtering
void BandFilter(float *data, int length, float lowFreq, float hiFreq, float
DataRate, int type);
  // 4. Notch filtering
void NotchFilter(float *data, int length, float Freq, float DataRate);
  // 5. Data binning: return a new length. After binning,
  DataRate/=BinWidth;
int BinData(float *data, int length, int BinWidth) {
  int c=0;
  for(int i=BinWidth; i<length−BinWidth; i+=BinWidth) {
    float tmp = 0;
    for(int j=−BinWidth; j<=BinWidth; j++) tmp += data[i+j];
    data[c++] = tmp / (BinWidth * 2 + 1);
  }
  return c;
}
int GetAvailableLength(int Length, int BinWidth) {
  return (Length − BinWidth−1) / BinWidth;
}
int GetAvailableBinWidth(int Length, int newLength) {
  return (Length −1) / (newLength + 1);
}
  // 6. Smooth Data
void DataSmooth(float *data, int length, int SmoothBin) {
  for(int i=0; i<length− SmoothBin; i++) {
    float tmp = 0;
    for(int j=0; j<= SmoothBin; j++) tmp += data[i+j];
    data[i] = tmp / SmoothBin;
  }
}
```

TABLE 9
Interface Security Checking

```
  // 1. Read serial number
int GetAmpSerialNumber( ); // DCamp unit
int GetBaseSerialNumber( ); // Interface unit
  // 3. Check if the use is legal
bool IsValid(int sNumber);
>>>>>>>>>>>>>>>>>>>>>>>>>>>>>>>>>>
```

Command List for Communicating with the USB Amplifier Interface:

The command list for the DC amplifier USB interface 66 is for the Cypress microprocessor that controls the communication between the DC amplifier 58 and the USB port connected to the user's PC 64. A sixteen (16) bit data communication structure is provided, so each data unit takes two bytes and so two bytes are assigned for each data unit, even though some times it is not necessarily required. In this list, the first two bytes are assigned for the command number. Since only a few commands are used currently, the second byte (Byte1) is reserved for the future and so is always “0”. The second two bytes are assigned for the communication data, and the third two bytes are for the address data. The two bytes of data are always the low byte first and then the high byte.

TABLE 10
Interface Commands

| Command | Byte0 | Byte1 | Byte2 | Byte3 | Byte4 | Byte5 | Byte6 | Byte7 | Byte8 |
|---|---|---|---|---|---|---|---|---|---|
| Reset | 0x00 | 0 | X | X | X | X | X | X | ? |
| ResetADC | 0x01 | 0 | X | X | X | X | X | X | ? |
| SingleADC | 0x11 | 0 | X | X | X | X | Chan | 0 | ? |
| ReqEndADC | 0x12 | 0 | X | X | X | X | Chan | 0 | ? |
| BatchADC | 0x13 | 0 | SRLo | SRHi | LenLo | LenHi | Chan | 0 | ? |
| ReqBatch | 0x14 | 0 | Block | Bl_Hi | X | X | Chan | 0 | ? |
| ContinueAD | 0x15 | 0 | SRLo | SRHi | X | X | Chan | 0 | ? |
| ReqStartCont | 0x16 | 0 | Bl_Lo | Bl_Hi | X | X | Chan | 0 | ? |
| ReqStopADC | 0x17 | 0 | X | X | X | X | Chan | 0 | ? |
| DigitalOut | 0x20 | 0 | DigiLo | DigiHi | X | X | X | X | ? |
| DigitalIn | 0x21 | 0 | DigiLo | DigiHi | X | X | X | X | ? |
| DigitalTrig | 0x22 | 0 | X | X | X | X | X | X | ? |
| ResetDC | 0x30 | 0 | X | X | X | X | X | X | ? |
| ReadDC | 0x31 | 0 | X | X | X | X | X | X | ? |
| SetDC | 0x32 | 0 | DCLo | DCHi | X | X | X | X | ? |
| ReadStatus | 0x33 | 0 | S_Lo | S_Hi | Chan | 0 | X | X | ? |
| ReadSN | 0x40 | 0 | X | X | X | X | X | X | ? |

Since DC amplifier 58 may lock up the USB interface 66, it should be checked every time before commands are sent. The value of amplifier status reflects whether the amplifier is working normally. Here are the possible situations:

| Value | Meaning |
|---|---|
| 0 | working normally |
| 4 | power not on (no response) |
| 5 | power too low |
| 3 | DC offset too high |
| 4 | DC offset too low |

Function list for the class communicating with the USB device:
I. Data Communication Functions
// 1. To request a single AD conversion:
bool LKCAnalogRequest( );
// 2. To obtain a single AD data:
bool LKCAnalogIn(ULONG &dataAddr);
// 3. To request a batch of AD conversion:
bool LKCAnalogInBatchRequest(long length);
// 4. To request a batch of AD conversion with trigger:
bool LKCAnalogInBatchTriggerRequest(long length);
// 5. To obtain a batch of AD data:
bool LKCAnalogInBatch(ULONG *dataAddr);
// 6. To send a single digital command:
bool LKCDigitalOut(UCHAR command);
// 7. To request a single digital data:
bool LKCDigitalInRequest( );
// 8. To obtain a single digital data:
bool LKCDigitalIn(ULONG &dataAddr);
// 9. To set DC offset to zero for the amplifier bool LKCSetDCOffset(0);
// 10. To get DC the amplifier status bool LKCGetAmpStatus( );
// Successful if the function returns 0, otherwise error (need an error message list).
II. Data Conversion and Memory Management Functions
// 1. To convert a single analog data
float ConvertADCData(ULONG data);
// 2. To convert a batch analog data
void ConvertADCData(ULONG *dataAddr, float *dataNew, long length);
float *ConvertADCData(ULONG *dataAddr, long length);
float *ConvertADCData(ULONG *dataAddr);
float *ConvertADCData( );
// 3. To apply memory for batch data
ULONG *SetADMemory(long length);
// 4. To apply memory for converted data
float *SetConvertADMemory(long length);
// 5. Destructor: release all memory
III. Parameter Setting Functions
// 1. To set sampling rate
int SetSampleRate(int rate); // return actual sample rate, not valid value if zero returned
// 2. To reset the DC offset
void SetDCOffset(float dcValue); // the DC value is stored in a float variable
// 3. To get current DC value
float GetDCOffset( ); // return the DC value
// 4. To set low-cut frequency
void SetLowCutFrequency(float lowCut);
// 5. To set hi-cut frequency
void SetHiCutFrequency(float hiCut);
// 6. To get low-cut frequency
float GetLowCutFrequency( );
// 7. To get hi-cut frequency
float GetHiCutFrequency( );
III. Data Processing Functions
// 1. Low-cut filtering
void LowCutFiltering(float *data, int length, float freq);
// 2. Hi-Cut Filtering
void HiCutFiltering(float *data, int length, float freq);
//3. Band width filtering
void BandFiltering(float *data, int length, float lowFreq, float hiFreq);
IV. Security Checking
// 1. Read serial number
int GetSerialNumber( );
// 2. Set serial number
void SetSerialNumber(int sNumber);
// 3. Check if the use is legal
bool IsValid(int sNumber);
>>>>>>>>>>>>>>>>>>>>>>>>>>>>>>>>>>>
The Advantages and Strategies of Using TI's ADS1255/1256 ADC Chip
Young Tsau Jul. 30, 2003

The Texas Instruments has released a new 24 bits A/D chip last month, which has the following features:
24 Bits, No Missing Codes
All Data Rates and PGA Settings
Up to 23 Bits Noise-Free Resolution
±0.0010% Nonlinearity (max)
Data Output Rates to 30kSPS
Fast Channel Cycling—18.6 Bits Noise-Free (21.3 Effective Bits) at 1.45 kHz
One-Shot Conversions with Standby Mode
Flexible Input multiplexer with Sensor Detect
Four Differential Inputs (ADS1256)
Eight Single-Ended Inputs (ADS1256)
Chopper-Stabilized Input Buffer
Low-Noise PGA
Self and System Calibration for all PGA Settings
5V Tolerant SPI-Compatible Serial Interface
Analog Supply: 5V
Digital Supply: 1.8V to 3.6V
Power Dissipation—As Low as 38 mW in Normal Mode, −0.4 mW in Standby Mode
   1. The advantages of using ADS1255/1256 to replace ADS1252/1254
   We currently use ADS1252/1254 A/D chip for our DC amplifier design. For each differential channel, we need to put a pair of buffer amplifiers for input impedance conversion, a fixed-gain differential amplifier, an impedance test circuit, and a DC offset adjusting circuit.
   The newly released ADS1255/1256 has the similar (a bit better) ADC resolution and fast-enough sample rate (up to 30 kHz), but more built-in circuits, including buffer amplifiers, a programmable gain differential amplifier, an open/short sensor detection circuit, and offset calibration circuit. It is possible that we can build the digital DC amplifier with minimum external components, therefore, to lower the power consumption and enhance the specifications.
   2. The Control of the ADS1255/1256
   The chip needs at least SCLK and DIN for input, and DOUT for data output. Optionally, DRDY, CS, and PWDN should also be controlled. Since we have to minimize the number of connections between the DC amplifier box and the interface unit in order to reduce the power consumption, the optimal number of the fiber optics connections is two: FOᵢₙ and FOₒᵤₜ. Since the data rate is up to 2.5M bps, the fiber optics transceiver must meet this specification.
   It seems to me that to have the best control of the chip operation, a microcontroller should be used. It is more powerful and flexible. Now Lin has found a powerful controller, PIC18F452, which meets all our requirements for the DC amplifier. It supports up to 10M instructions per second, and the following is the assembly code for taking one data that takes about 20 uS based on 40 MHz clock: [image] [image] [image] [image] [image] [image] [image] [image] [image]
   3. The Consideration of Channel Switching
   The channel switching for ADS1255/1256 is a bit too slow for our data acquisition purpose, although a single ADS1256 supports 4 differential channels. It seems to me that we should use only one differential channel and use 2 or more chips to achieve the multi-channel scheme. Each chip dissipates about 13 mA in active mode and less than 1 mA in standby mode. Therefore, the power consumption should not be a problem. A microcontroller should be able to control all the chips in a nice way that all the optional control pins can be individually controlled.
   In the applications where multiple channels (>8) are involved, we may consider using all 8 single ended channels for each ADS1256. If we use 4 chips for the high speed 4 differential channels analog inputs, we have potentially 32 single ended channels at the switch speed roughly at the sample rate (about 1 msec). This time shift is tolerable for EEG recordings, which requires sample rate about 100 Hz. The EEG amplifier has much wider clinical applications than ERG/EOG/VEP, as it is essential for neurologists.
   4. The Impedance Test Performance
   The impedance test is carried out by applying an alternating constant current source to the differential input and measuring the amplitude at the input. Ideally, the applied alternating constant current source to the input should be from positive to negative to avoid potential polarization at the electrode connection points. However, ADS1255/1256 can only provide a single ended on-off connection to the current source, resulting pulse type current application to the input instead of alternation type. This will still measure the impedance, but may not be as accurate as the alternation one.
   5. The Communication Between DC Amplifier Unit and the Interface Unit
   The DC amplifier unit receives control commands from the interface unit, and sends out the data upon request. Here is the control command list:
   Using PIC18F452 for DCamp Unit and Interface Unit Controller
   Young Tsau Aug. 12, 2003
   Currently, we utilize EZ-USB series 2100 (CY7C46xx, Cypress Co.) for our new DCamp computer interfacing. It has the following features:
Built-in Enhanced 8051 @ 16 MHz clock
8 KB RAM
40 pins
8/16-bit Databus|GPIO|I2C| Memory Expansion Port, up to 56K bps UART communication
USB 1.0 interface
   It can be connected to a computer via a USB cable, which also provides the power supply. However, there are a number of features that this chip does not meet our requirement:
Relatively slow operation (16 MHz)
Relative slow serial port communication (<56K bps)
Limited program memory
   The data rate from the DCamp unit to the interface unit is calculated as follows:
   Four channels at the sample rate 20 kHz generate data at 4×20×3=240 kbytes/sec, that is about 2.4 Mbits/sec when communicating via a serial port (8 bits plus start and stop bits). Only serial port communication should be used because the fiber optics is used for the connection between DCamp unit and interface unit.
   Unless the data is buffered in the DCamp unit, the data cannot be sent out to the interface unit via a 56K serial port. Because the time is very tight for the DCamp unit controller (PIC18F452), there is no time left for it to buffer the data and transfer later and this may generate peripheral device synchronization problems.
   Ideally, the interface unit buffers all the data and controls all peripheral devices such as ganzfeld, mini-ganzfeld, and so on. The Cypress microcontrollers just cannot achieve the speed for serial port communication. Therefore, we need to find a better microcontroller to meet our requirement.
   PIC18F452 from Microchip Co. is a high end microcontroller that has the following features:
C compiler optimized architecture/instruction set, compatible with the PIC16 and PIC17 instruction sets
Linear program memory addressing to 32 Kbytes
Linear data memory addressing to 1.5 Kbytes
Up to 10 MIPs operation: DC −40 MHz osc./clock input
16-bit wide instructions, 8-bit wide data path
Timer0, Timer1, Timer2, and Timer3
Master Synchronous Serial Port (MSSP) module, supporting 3-wire SPI and I2 modes, USART RS-485 and RS-232, and Parallel Slave Port (PSP)
Programmable Low Voltage Detection (PLVD)
100,000 erase/write cycle Enhanced FLASH program memory typical 1,000,000 erase/write cycle Data EEPROM memory
Self-reprogrammable under software control
Single supply 5V In-Circuit Serial Programming via two pins
In-Circuit Debug (ICD) via two pins
Low power, high speed FLASH/EEPROM technology
Fully static design
Wide operating voltage range (2.0V to 5.5V)
Industrial and Extended temperature ranges
Low power consumption: <1.6 mA typical @ 5V, 4 MHz; <0.2 μA standby
   It is much faster (up to 40 MHz clock and 10 MHz instruction operation), fast serial port communication (3-wire asynchronous USART up to 2.5M bps), and 32 Kb linear program memory. Therefore, PIC18F452 seems to be a better choice for our design.
   However, PIC18F452 doesn't have a USB port. This architecture changes our strategy of communication between DCamp and interface units. (USB 1.0 has some limitations itself. For example, its highest time resolution is 1 msec but we desire higher than this.) Currently, our solution is to use an ECP (extended capability parallel) communication port, which is also known as printer port for most computers. It has much higher time resolution (nsec) and faster data transfer rate (about 5 Mbytes/sec). Our design using PIC18F452 for the interface unit has three communication ports:
High speed 3-wire serial port (2.5M bps)
High speed ECP port (5 Mbyts/sec)
Slow 5-wire serial port (9600 bps)
   The 3-wire serial port utilizes pin RC6 and pin RC7. Since the communication is made between two PIC18F452 controllers running at the same clock speed (40 MHz), there should be no matching problem.
   The ECP port will utilize an 8 bits bidirectional port, 4 output lines, and 4 input lines. It also requires at least one input line for the external interrupt, in order to respond to the input data in time.
   PIC18F452 has only one formal serial port, which is used by the high speed 3-wire communication between the DCamp and interface units. We need to utilize plain I/O pins to program into serial ports. The following C code is an example of using D0 and D2 as Tx/Rx port:
   In addition, I'd like to have trigger in and trigger out lines embedded into the slow serial port, which gives more control over the peripheral stimulation devices.
   A potential problem I have now for using PIC18F452 is its limited RAM for temporary data storage. I originally had an idea to be able to store up to 64 Kbyts data in the interface unit. The current answer to this limitation is to transfer all data instantly to the host (computer) and let the computer do the storage and all signal processing. Since the design is stackable (more features can be added on to next stage board of interface unit), we may later design the next stage board with more memory and USB feature. In the future, it is believed that PIC18F series will have USB port implemented, as USB is becoming an industrial communication standard. I would expect that we can add these feature in the near future.
   >>>>>>>>>>>>>>>>>>>>>>>>>>>>>>>>>>>
   Using Single Fiber Optics Cable to Carry out Bi-Directional Communication:

The idea of using fiber optics for digital data communication between DC amplifier unit 58 and the interface unit 62 is described as follows:
1. Safety. There is no risk of leakage current from any other device to the DC amplifier unit powered by a low voltage battery when they are connected via fiber optics.
2. Noise reduction. Any disturbance of power source, 60/50 Hz interference, and so on will not reach the DC amplifier unit via fiber optics.

The fiber optics communication needs to be bidirectional: DC amplifier unit 58 receives commands from interface unit 62 and sends out data to the interface unit. In principle, two fiber optic cables are usually required to carry out the task. There is a potential risk of misplacement of input/output cables to the wrong jacks. In addition, two jack exposures on the DC amplifier box look bulky (each one takes about 10 mmˆ2 space). Finally, parallel fiber optics cables are more expensive.

For these reasons, a single line of fiber optics cable will provide bidirectional communication, because a single fiber can be used for passing signal light in both directions. The real challenge is to separate the input/output signals inside the DC amplifier unit 58.

A pair of inexpensive fiber optic signal splitter of the type available from Radio Shack are used for separating one optical signal source into two cables. Each splitter was opened and used in configuration illustrated in FIG. 12, showing transmit and receive junctions connected to a fiber optic jack for attachment to a bidirectional fiber-optic cable. Epoxy is used for gluing the optic fibers to the transmission and reception diodes.

It will be appreciated by those of skill in the medical arts that the present invention makes a very accurate sensing system available for use in detecting and measuring neural response signals. For purposes of nomenclature, the term “neural response signals” is construed to mean and include any biologically occurring electrical signal, such as those signals mentioned above. The term “amplifier” shall be construed to mean and include any active circuit element or combination, of discrete components or as portions of an integrated circuit, performing impedance matching, filtering or gain adjustment, and the embodiments illustrated above are merely intended to be illustrative of workable prototypes that have been demonstrated in the applicant's work.

Having described preferred embodiments of a new and improved method, it is believed that other modifications, variations and changes will be suggested to those skilled in the art in view of the teachings set forth herein. It is therefore to be understood that all such variations, modifications and changes are believed to fall within the scope of the present invention as defined by the appended claims.

### APPENDICES

The attached appendices A-D include additional disclosure:

Appendix A, entitled “Preliminary Results” includes empirical data on the performance of the amplifier of the present invention.

Appendix B, entitled “Proposal” includes additional disclosure on the amplifier of the present invention.

Appendix C, entitled “Interface Program Listing” includes additional disclosure on the amplifier of the present invention, and is included on the enclosed CD-ROM.

Appendix D, entitled “Interface Program Listing” includes additional disclosure on the amplifier of the present invention, and is included on the enclosed CD-ROM.

### APPENDIX A

Preliminary Results

Design of the Electronic Circuits

A complete digital DC amplifier system includes i) an isolated and battery powered patient amplifier unit which has the output in digital format, ii) an interface unit that transceives the control commands from a host computer to the amplifier and other peripheral devices, and the data from the amplifier to the computer, and iii) computer drivers which communicates an application program running on a computer (PC, MAC, etc.) to the amplifier and other peripheral devices through the interface unit.

1. The Analog Signal Input Circuit

The amplifier and ADC are connected as true differential to maximize CMNRR. All the high speed delta-sigma ADC chips have been designed with differential analog input, such as ADS1252, ADS1254, and ADS1256 from Texas Instruments. They can be directly connected with the true differential instrumental amplifiers that have both differential input and output, such as TH4130 from Texas Instruments. The consideration of minimum number of the amplifier stages is emphasized in order to minimize the internal noise, and also to lower the power consumption.

[image]

2. Voltage Reference Circuit

The ADC chip needs a voltage reference for carrying out digitized data refering to the reference. Since the ADC is extremely sensitive to the small voltage fluctuation at uV level, the reference needs to be precise and noise free. Large capacitance and low equivalent series resistance (ESR) capacitors are used on the voltage reference inputs. The following circuit has been recommended by TI for ADS1252:

[image]

3. Control Circuit

Many parameters about the digital DC amplifier are programmable, such as the input buffer, amplification gain, DC offset, amplifier calibration, the number of active channels, the sampling rate, and the power management. As the amplifier/ADC is controlled by complicated data transfer and sequential command procedures, it is necessary to use a microcontroller to manage the data communication and parameter setting tasks. The following considerations have been taken into account in choosing the microcontroller: the processing speed, power consumption, simplicity of data communication, data communication speed, and sufficiency of control ports. We have chosen PIC18LF452 (Microchip, Inc.), which supports up to 10 mips to achieve the data transfer rate up to 30 ksps per channel (that is 90 kilobytes/sec), less than 10 mA current at 3.3V power supplier, high speed (up to 2.5 mbps in full duplex asynchronous mode and 10 mbps in half duplex synchronous mode) three wire serial communication (In, Out, and GND) scheme, and up to 33 programmable bidirectional I/O ports. It also has 32 K bytes flat program memory and 1526 bytes RAM.

[image]

The amplifier unit receives commands from and sends data to a user's computer (PC or MAC) via an interface unit, which serves as a controlling center for various I/O and data buffering. PIC18LF452 has been chosen as it simultaneously supports high speed serial communication that is used for data acquisition, a general purpose 8 bits parallel port and a 9600 bps RS232 serial port, and a hardware handshaking parallel port for ECP/USB data communication to the host computer.

[image]

The high speed serial communication is made via an interface of a single optical fiber cable to eliminate external interference noise and to maximize the safety. The half duplex communication method has been applied to simplify the hardware scheme. Therefore, the interface includes a current driver for the high speed LED and a photodiode with detection comparater circuit on both sides of amplifier unit and interface unit, and a single pass optical fiber cable, as shown below:

[image]

4. Power Supply Circuit

The patient amplifier unit is powered by a 7.2V Lithium-Ion rechargeable battery to minimize the noise introduction and to maximize the patient safety. The amplifier unit uses two voltages (3.3V and 5V), which are obtained by using two linear regulators, as most switching DC/DC converters generate extra high frequency noise at uV level. During recharging time, the patient amplifier unit is internally disconnected by using a reed relay for safety and noise considerations. As one charge of battery should last at least the normal working hours (8 hours), the battery's capacity (mAh) should be larger than 8× circuit current consumption (mA).

The current for the pre-amplifier and A/D converter is quite low, around 15 mA during data conversion and lower (<5 mA) during quiescent, and the microcontroller takes about 10 mA. The other circuits consume less than 5 mA. However, the fiber optics device can take up to 25 mA during data transfer, which is the major power consumption component if high speed constant data transfer is in use. In our case of ERG/EOG/VEP recordings only a few minutes of medium speed data transfer per patient is involved, the total power consumption would be less than 100 mA and the battery should have 800 mAh capacity for 8 hours continuous use.

[image]

For the circuit shown above, the currents at different conditions are measured and shown as follows:

(Current Measurement Comparison Table)

5. Considerations of Circuit Layout

Since the amplifier unit is extremely sensitive to the external noise at the level of uV, the layout of the components on the analog side and even the positioning of the connection wires need to be carefully considered. The power supplier and ground are separated between analog and digital sides with a single node connection. Inductors have been used for blocking high frequency bypassing. The digital part of the circuit layout does not have special requirement.

Programming the Controllers

Both the amplifier unit and interface unit utilize one or more microcontrollers, the firmware of which is programmed using hybrid C and Assembly languages. PIC18LF452 has been chosen as the controller, which is a high speed processor that can process up to 10 mips at the power consumption less than 10 mA. It is still a challenge of speed when high speed (30K sps) ADC is selected, where the operation code must be written in Assembly language to achieve the speed. Most other operations would be designed using C language since it is fast enough (<10 uS response time) and the processor has way enough program memory (32 KB) for less efficient destination code generation from the C source code, comparing to the high-efficiency but cumbersome Assembly code.

1. The DC Amplifier Unit

Initializing ports, interrupts, and parameters. The PIC18LF452 processor has 33 programmable bi-directional I/O ports that can be assigned as input or output ones. They need to be initialized at the beginning when the power is turned on. The serial communication receiving port is assigned with an interrupt that needs to be initialized at power-on time. Other parameters, such as gain, sample rate, and number of active channels, are also initialized to the default values at power-on time. All other unused interrupts are disabled.

Starting a real time clock and power management. Once the power of the DC amplifier unit is turned on, a real time clock is started to count the idle time when there is nothing going on except for waiting for the incoming commands. The unit will enter the standby mode if the idle time is beyond 10 minutes (adjustable by the control command) to save the power. Any incoming command wakes up the unit. The unit can also be brought to the standby mode or waken up by the incoming commands.

Parsing the incoming commands. Many parameters are adjustable by the user through the host computer to meet different applications. They are categorized in amplifier settings, the number of active channels, the ADC settings, the read-back of current settings, and data acquisition. The incoming command is first parsed and then processed by the corresponding routines.

Setting up the amplifier. The amplifier input can be set as either differential or single-ended mode. The gain can be set to ×1, ×2, ×4, ×8, ×16, ×32, or ×64. The DC offset can be calibrated.

Setting up the active channels. Any individual channel can be activated or de-activated, resulting a combination of active channels. As the new setting of active channels may affect the highest available sample rate, the current sample rate setting is checked and may be modified every time when there is a change of channels.

Setting up the ADC parameters. There are two major parameters for ADC data acquisition: sample rate and data length. The sample rate is pre-defined by the ADC chip itself that can be one of the following values: 30K, 15K, 7.5K, 5K, 2K, 1K, 500, 200, 100, 60, 50, 30, 25, 10, 5, 2.5 sps. They have −3 dB cut-off high frequency at 10K, 5K, 2.5K, 1.75K, 660, 330, 175, 66, 33, 20, 17, 10, 8, 3, 2, 1 Hz, respectively. The data length can be either predefined (256, 512, 1024, 2048, 4096, 8192, 16K, 32K, 64K) or any size.

Requesting the current settings. Since the settings may be modified during parameter setting (see above) or the unit might be reset during operation, the host computer needs to verify the settings before starting data acquisition. The parameter data are sent back to the host computer within half msec at the speed 2.5 mbps or above.

Data acquisition. Within 10 uS after receiving the data acquisition command from the host computer through the interface unit, the digitized data are acquired based on the settings of the amplifier unit, and are sent back to the host in real time manner. Since this is time sensitive, the code has been written in Assembly. When the data length is set to 0, the data will be acquired and sent continuously until an incoming command stops it.

The logic flow charts of the amplifier unit are listed as follows:

[image]

[image]

[image]

2. The Interface Unit

Initializing the ports, interrupts and parameters. The 33 programmable bi-directional I/O ports are initialized at the beginning when the power is turned on, according to their assignments. The serial communication receiving port is assigned with an interrupt that needs to be initialized at power-on time. One of the thrce line interrupts is assigned for ECP/USB transceiver data communication, one is assigned for the low speed (9600 bps) serial port, and another one is assigned for the parallel port. All other unused interrupts are disabled. The data buffer of 1024 bytes is assigned out of the total 1526 bytes of RAM.

Parsing the incoming commands. They are categorized in those setting the amplifier unit, requesting the setting values, tranceiving data between the host computer and the parallel/serial port. The interface unit itself does the minimum processing work and only passes the commands to the peripheral devices and sends data back to the host computer.

Synchronizing the data acquisition and trigger. It is essential for the data acquisition and a stimulation device to be synchronized for the recording accuracy. The time resolution of synchronous is defined as at most the interval between two acquired data at the highest sample rate (30K sps), or less than 34 uS. A trigger out signal is sent by the interface unit every time when the data acquisition starts. In the trigger in mode, the data acquisition command is on hold until the trigger in signal is received or the operation is aborted.

Buffering the acquired/setting data and sending back to the host computer. Since the host computer with a multi-task operating system like microsoft ME/NT/2000/XP has 1 msec minimum response time resolution, the data must be transferred in burst mode when the data transfer rate is higher than 1 Kbytes/sec (sample rate is higher than 200 Hz for a single channel input), and so it needs to be buffered before sent out.

The logic flow charts of the interface unit are listed as follows:

[image]

[image]

[image]

[image]

Programming the Driver on the Host computer Side

Although it is possible to directly access the amplifier in an application program when it is connected to a host computer via an ECP port, a interface driver will generally ease programming applications, especially if it is connected via a USB port. For example, the LKC's ERG/EOG/VEP test application programs running on a PC typically access all the peripheral devices via an interface driver, which is a dynamic linking library (DLL) program communicating to a hardware abstract layer (HAL) driver. Furthermore, the acquired data in the original 3 byte integer format with no unit needs to be converted to float type data for direct measurement and for example, may need to be filtered. These are processed on the host computer as it is much more powerful and resourceful. Therefore the complete DC amplifier as a product would include not only the hardware (amplifier unit and interface unit with the firmware) but also the software (the interface driver).

The driver has the following functions: setting the DC amplifier, setting the peripheral devices including the stimulator, and acquiring the data. The digital signal conditioning and processing functions are also implemented in the driver as a part of the system. All the functions are declared in a C header file that the user can include in the program for calling them.

The DC amplifier parameters are set by calling the functions with the corresponding easy-recognizable names instead of directly sending out the command code. These functions are summarized as follows:

// 1. To set sampling rate

int SetSampleRate(int rate); // return actual sample rate, not valid value if zero returned

int GetSampleRate( );

// 2. To set data length

int SetDataLength(int length); // return actual length

int GetDataLength( );

// 3. To set channel

void EnableADFlag(UCHAR *FlagChan, bool enable);

void EnableADCChan(int channel, bool enable);

bool GetEnabledChannels(UCHAR *FlagChan);

// 4. To set input mode void SetAmplifierMode(bool SingleEnded);

bool GetAmplifierMode( );

// 5. To set amplifier gain void SetAmplifierGain(UCHAR chip, UCHAR gain);

UCHAR GetAmplifierGain(UCHAR chip);

// 6. Calibrations void CalAmpGain( );

void CalAmpOffset( );

void CalAmpSystem( );

// 7. To get the amplifier and ADC status bool GetAmpStatus(UCHAR &status);

bool GetCurrentADLength(int &length);

// 8. To put the amplifier in standby mode and wake it up bool PowerSaveMode( ); bool WakeupMode( );

// 9. To reset the amplifier and interface:

bool AmplifierReset( );

bool InterfaceReset( );

The driver also facilitates the host computer to communicate with peripheral devices connected to the parallel or serial port on the interface unit. Accessing the interface unit parallel and serial ports as well as the trigger ports is made by calling the following functions:

// 1. To send a single digital command via the interface unit serial port:

bool SerialOut(UCHAR command);

// 2. To request a single digital data via the interface unit serial port: bool SerialIn (UCHAR &data);

// 3. To send a single digital command via the interface unit serial port: bool ParalOut(UCHAR command);

// 4. To request a single digital data via the interface unit serial port: bool ParalIn (UCHAR &data);

// 5. To set periodic trigger out in msec bool SetTriggerOutPeriodic(int interval);

// 6. To Start periodic trigger out

bool StartTriggerOutPeriodic( );

// 7. To stop periodic trigger out bool StopTriggerOutPeriodic( );

// 8. To check trigger in bool CheckTriggerIn(BOOL in);

// 9. To set trigger in call back bool CallBackTriggerIn(void *CallBackAddress);

// 10. To send a trigger out signal bool SendTriggerOut( );

// 11. To set trigger signal polarity bool SetTriggerInPolarity(BOOL pos);

bool SetTriggerOutPolarity(BOOL pos);

There are several protocols for starting the data acquisition: simple start, start with trigger out, start with synchronous periodic trigger out, and start with trigger in. Each one has its indications of use. LKC's ERG/EOG/VEP test programs utilize all the protocols.

// 1. To obtain a single AD data: bool AnalogIn(float &dataAddr);

// 2. To request a batch of AD conversion:

bool AnalogIn(long &rate, long &length, float *dataAddr);

bool AnalogIn(long &rate, long &length, float *dataAddr, int chan);

// 3. To request a batch of AD conversion with trigger out:

bool AnalogInBatchTrigger(long &rate, long &length, float *dataAddr);

bool AnalogInBatchTrigger(long &rate, long &length, float *dataAddr, int chan);

Once the acquired data are received, they are stored in the memory address passed and allocated by the user. The data is converted to double precision float type in the unit of uV from its original type of integer format. The data can also be binned to generate various “sample rate” with even higher SNR. For example, the binning with a width of 3 for the data acquired at 30K sps generates a new data set of 10K sps with ⅓ of its original data length.

For the filtering functions, the low-cut, high-cut, and 50/60 Hz notch filers are commonly used and so are built in the driver. In addition, a DC offset removing (so called baseline correction) function and a smoothing function are also useful in data conditioning and analysis. The functions are listed as follows:

// 1. Low-cut filtering

void LowCutFilter(double *data, int length, float freq, float DataRate, int type);

// 2. Hi-cut filtering

void HiCutFilter(double *data, int length, float freq, float DataRate, int type);

// 3. Band width filtering

void BandFilter(double *data, int length, float lowFreq, float hiFreq, float DataRate, int type);

// 4. Notch filtering

void NotchFilter(double *data, int length, float Freq, float DataRate);

// 5. Data binning: return a new length. After binning, DataRate/=BinWidth;

int BinData(double *data, int length, int BinWidth);

int GetAvailableLength(int Length, int BinWidth);

int GetAvailableBinWidth(int Length, int newLength);

// 6. Smooth Data

void DataSmooth(double *data, int length, int SmoothBin);

// 7. Baseline correction

double BaselineCorrection(double *data, int length);

The Test Results

The DC amplifier has been tested under two conditions: the input open or shortened for measuring the DC amplifier's internal noise level and connected to a sine wave signal source for measuring dynamic range and frequency response characteristics.

Noise Level Analysis.

Root mean square (RMS) and peak-to-peak (p-p) amplitude have been used as measures of internal noise. RMS and p-p are defined as

[image]

[image]

[image]

[image]

[image]

[image]

[image]

[image]

[image]

[image]

[image]

[image]

[image]

[image]

[image]

[image]

[image]

[image]

[image]

[image]

The DC amplifier is not sensitive to the input open condition. There was a slight decrease of noise level when the inputs were shortened. The data were acquired at 1K sps and at 100 sps, respectively. Each data set had 256 samples. No analog or digital filtering was applied to the data acquired from the DC amplifier. The noise level for the 1K sps data set was about 0.39 uV RMS (2.57 uV p-p), and that for the 100 sps data set was about 0.09 uV RMS (0.61 uV p-p) (see figures below).

[image]

The noise level was also analyzed on Grass Instrument's Model 12 amplifier as well as LKC's programmable amplifier. Since they don't have the exact same frequency settings with both no DC selection, the low-cut and high-cut frequencies were set as close as possible to make the data be comparable to the DC amplifier. The inputs were always shortened, otherwise the noise level was much higher for both amplifiers, often contaminated with 60 Hz noise. The data were acquired by using a 12 bits ADC card (PCI-DAS1200, Measurement Computing Corporation) at sample rates of 1000 sps and 100 sps, respectively.

Model 12 amplifier. The bandwidth was set to 0.1-1000 Hz for the sample rate 1000 sps, and the bandwidth set 0.1-100 Hz for 100 sps. The gain was set to ×500K. The noise level was measured from the data sets with 256 samples each. The noise level for the 1K sps data set was about 2.0 uV RMS (13.2 uV p-p), and that for the 100 sps data set was about 1.05 uV RMS (6.9 uV p-p) (see figures below).

[image]

The amplifier was saturated if the inputs were open at the gain ×500K. In order to measure the noise level for open input, the gain was adjusted to ×50K, while the noise was still tremendously higher than shortened input mode (see figure below).

[image]

LKC's amplifier. The bandwidth was set to 0.05-1500 Hz for the sample rate 1000 sps, and the bandwidth set 0.05-100 Hz for 100 sps. The gain was set to ×400K. The noise level was measured from the data sets with 256 samples each. The noise level for the 1K sps data set was about 2.36 uV RMS (15.6 uV p-p), and that for the 100 sps data set was about 0.9 uV RMS (6.0 uV p-p) (see figures below).

[image]

In conclusion, the DC amplifier has more 5 times lower noise level at 1000 sample rate and 10 times lower noise level at 100 sample rate than Grass's Model 12 and LKC's amplifier. The noise is further reduced when digital filtering is applied. Furthermore, the DC amplifier has much higher resistance to the external noise, as the noise level does not dramatically change whether the inputs are shortened or open, indicating a much better CMNRR.

Dynamic Range.

The dynamic range of the DC amplifier was measured by connecting to a p-p 100 mV 100 Hz sine wave with +0.5V DC offset generated by a function generator (Goldstar FG-8002). A trial of 2048 samples was acquired at 30K sps. The same data is plotted in three scale ranges to show the outline of the recording, the outline of the sine waveform, and the details of the curve at a sine wave peak. The curve details are in the range of 0.5 mV and still smooth comparing to the internal noise level. Therefore, the sine wave signal with 100 mV p-p amplitude was faithfully recorded even though a 0.5V DC offset was present, showing that the dynamic range of the amplifier would satisfy the data acquisition of small AC signals with big DC offset and the amplifier does not need further DC adjustment (see figures below).

[image]

[image]

To further measure the noise level of the analog input, the same signal source was then attenuated 10 dB. This resulted in a signal source of 10 mV with 50 mV DC offset. Although the sine wave was still faithfully recorded, the details were dominated by the background noise of the function generator (RMS is about 25 uV), which is much higher than the amplifier's internal noise (see figures below).

[image]

Frequency Responses.

The DC amplifier was also connected with signal source of various frequencies to test the frequency response characteristics. The measure was made with sine wave signal at 2 Hz, 20 Hz, 200 Hz, and 2000 Hz. The amplitude of signal at each frequency was calibrated with an oscilloscope (Tektronix 2215, DC-60 MHz). All the data were acquired at 30K sps. Recordings are plotted in the following figures:

[image]

[image]

The amplitude of the acquired data for lower frequencies is faithful to the analog signal comparing to the display on the oscilloscope. It starts losing the amplitude at the frequencies higher than 2000 Hz (about 10%). According to the data sheet for ADS1252/1254/1256 provided by Texas Instruments, the frequency response has the following curve:

[image]

In other words, its −3 dB attenuation is around 5 KHz. This satisfies most biomedical signals frequency ranges. For the slower frequency signals, the amplifier records faithfully.

What is claimed is:

1. A wearable medical instrument amplifier enclosed within a portable housing for processing a patient's or subject's neural response signals, comprising:
(a) a balanced input circuit adapted to receive first and second sensing electrodes conducting first and second balanced input neural response signals;
(b) a balanced buffer amplifier stage having balanced inputs responsive to said first and second balanced input neural response signals, said balanced buffer amplifier stage generating first and second buffered neural response signals therefrom;
(c) a balanced gain-adjustable pre-amplifier stage having balanced inputs responsive to said first and second buffered input neural response signals and having a control input adapted to receive a gain control signal; said balanced gain-adjustable pre-amplifier stage generating first and second pre-amplified neural response signals therefrom;
(d) an analog to digital converter having greater than 20 bits resolution, being capable of selectable sample rate and having balanced inputs responsive to said first and second pre-amplified neural response signals; said analog to digital converter generating a digitized neural response signal therefrom;
(e) a microprocessor programmed to receive and store said digitized neural response signal and, in response thereto, generating a gain control signal for said gain-adjustable pre-amplifier stage and generating neural response signal data for analysis and display.

2. The wearable medical instrument amplifier of claim 1, further including:
(f) said microprocessor being further programmed to generate an impedance test signal; and
(g) a balanced output impedance test amplifier stage having an input responsive to said microprocessor impedance test signal; said balanced output impedance test amplifier stage generating first and second impedance test signals in response.

3.
